(19) United States
(12) Patent Application Publication
(10) Pub. No.: US 2015/0080920 A1
(43) Pub. Date: Mar. 19, 2015

(54) DEVICE FOR PREPARING A HOLE FORMED IN A BODILY WALL AND SYSTEM FOR MAKING AN ANASTOMOSIS BETWEEN A BODILY WALL AND CONDUIT

(71) Applicants: Arnold Ferlin, (US); VETAGRO-SUP INSTITUT D'ENSEIGNEMENT SUPERIEUR ET DE RECHERCHE, Marcy L'etoile (FR)

(72) Inventors: Arnold Ferlin, Lyon (FR); Claude Carozzo, Dommartin (FR)

(21) Appl. No.: 14/383,436

(22) PCT Filed: Mar. 11, 2013
(86) PCT No.: PCT/FR2013/050509

§ 371 Date: Sep. 5, 2014

(30) Foreign Application Priority Data
Mar. 9, 2012 (FR) 1200731

(51) Int. Cl. A61B 17/11

(52) U.S. Cl. CPC A61B 17/11; A61B 2017/1103; A61B 17/1114; A61B 17/0401
USPC 606/153

(57) ABSTRACT

The disclosure relates to a system for preparing an opening created on an organic wall, comprising: —a supporting body defining a longitudinal axis and having a proximal portion and a distal portion adapted for insertion through the opening; —external clamping elements deployable in an outwardly flaring arrangement relative to the supporting body and able to cover portions of an annular surface of said wall; and—biasing elements arranged to tighten and juxtapose said external clamping elements on said body in order to progressively deform said annular surface so as to shape on said wall a tubular portion extending along the longitudinal axis and form an anchoring clamp consisting of said external clamping elements.

### CROSS-REFERENCE TO RELATED APPLICATIONS

This application claims priority under 35 USC §119 and the Paris Convention to French Patent Application No. FR 12 00731, filed on Mar. 9, 2012.

### FIELD OF THE DISCLOSURE

The present invention relates to the field of surgery, and in particular to the fields of urological, visceral, vascular, and other surgeries.

More particularly, the invention relates to a surgical treatment system for performing an anastomosis between an organic wall having an opening and an organic hollow tube, such as the bladder and urethra of a patient. The invention may, for example, be used following a prostatectomy undergone by the patient, typically a radical prostatectomy or total vesiculo-prostatectomy, with the aim of surgically restoring communication between the bladder and the terminal opening of the urethra at the end opposite the urethral meatus.

### BACKGROUND OF THE DISCLOSURE

Conventionally, anastomosis between the bladder and urethra of a patient is performed manually by surgeons using a needle to place one or more sutures. During such a procedure, the surgeons must first reconstruct the bladder neck. To do so, they deform the bladder wall at the opening created in this wall by the prostatectomy, to form a neck or tubular portion to connect to the urethra by suturing.

Such a treatment method offers certain advantages, including the fact that it creates an end-to-end connection of the urethra and the neck or tubular portion formed on the bladder wall. More specifically, such a method directly joins the edge of the bladder neck and the edge of the terminal end of the urethra, without having to fold either of these edges back. Direct contact is thus established between the mucosa of the respective edges, which is particularly advantageous in terms of healing and a complete and reliable restoration of communication between the patient's bladder and urethra.

However, this manual anastomosis technique has many disadvantages and is particularly difficult to perform. Such a technique requires a particularly long period for the surgery and a high level of dexterity in the surgeon, especially for reconstructing the bladder neck and precisely aligning the ends of the two channels to be connected, which in this case are the terminal opening of the urethra and the neck or tubular portion shaped on the bladder wall. Such a manual reconnection is made even more complex because the procedure area, located behind the pubic bone, is particularly difficult to access and contains the sphincter whose integrity must be preserved throughout the procedure by avoiding any pulling, perforation, squeezing, etc.

Moreover, this manual surgical method can have the disadvantage of being performed as open surgery and requiring large incisions in the body wall in order to access the procedure area, resulting in many inconveniences for the patient, particularly in terms of length of hospitalization, recovery time, and postoperative sequelae.

Finally, because of the manual nature of this surgical technique it is relatively tedious for surgeons, who must pay particular attention to suturing a properly tight anastomosis and establishing a consistently tight connection along the circumference of the anastomosis area. An insufficiently tight connection between the two portions of organic tubes hinders tissue healing, poses a risk of infection, and may ultimately result in separation of the connection between the two portions to be joined. Conversely, an excessively tight connection can cause tissue damage and lead to stenosis in the anastomosis area.

Another known technique is described in document US 2008 114385A and consists of folding the edge of the bladder neck and the edge of the terminal opening of the urethra inwards or outwards, and attaching the folded portions by a series of staples introduced from inside or outside the hollow organs to be treated. However, such a technique is not completely satisfactory because, since the edges of the openings connecting the two bodies have been folded, the connection between these two bodies is not end-to-end and their respective mucosa are not touching. This does not allow a natural consolidation of the anastomosis and requires long term fasteners, thus increasing patient recovery time, in particular bladder function, and can lead to the formation of bladder stones. Furthermore, the folding of organ portions to establish a connection creates a risk of thrombosis or stenosis at the anastomosis when these portions are folded inside the organs to be treated, and a risk of infection when these portions are folded outside the organs to be treated, where their mucosa may be exposed to a bacteria-rich environment. Moreover, one can easily see that this technique requires a significant number of staples covering a large portion of the periphery of the anastomosis area, and made of non-resorbable material to ensure longevity of the anastomosis as the tissues heal.

Finally, for an anastomosis between an organic wall having an opening and a organic hollow tube, such as the bladder and urethra of a patient, in particular after a prostatectomy, a first problem consists of forming a tubular portion on the wall at the opening, and giving this tubular portion a geometry that allows it to fit precisely against the end of the organic tube and allows establishing contact between their respective mucosa. The opening formed in the wall during the prior surgery may not necessarily have a regular geometry, and the wall may vary in thickness along the circumference of the opening: establishing a snug fit between the wall and hollow tube and mating their respective mucosa can be particularly difficult. None of the above techniques offer a satisfactory solution to this problem.

### OBJECTS AND SUMMARY OF THE DISCLOSURE

According to a first object, the present invention provides a system for preparing a created opening on an organic wall, particularly in preparation for an anastomosis with a organic hollow tube, in order to form on the wall at the opening, in a reliable and reproducible manner, a tubular portion having a predetermined geometry.

The invention further aims to provide a mechanical system which provides surgeons with the ability to perform procedures in a minimally invasive manner, in particular via the natural passages of the patient, in order to limit operative trauma, and which allows preparing an opening in a wall of an organ or vessel in a reliable and reproducible manner, particularly in preparation for a subsequent anastomosis with a organic hollow tube or a prosthesis such a vascular prosthesis.

For this purpose, a first aspect of the invention relates to a system for preparing a created opening on an organic wall, remarkable in that it comprises:
a supporting body defining a longitudinal axis and having a proximal portion and a distal portion, said distal portion being adapted for insertion through the opening;
external clamping elements deployable in an outwardly flaring arrangement relative to the supporting body while being distributed circumferentially, and able to cover, about said opening, portions of an annular surface of said organic wall; and,
biasing elements arranged to tighten and juxtapose said external clamping elements on said supporting body in order to progressively deform said annular surface so as to, on the one hand, shape on said organic wall a tubular portion extending along the longitudinal axis, and on the other hand, form an anchoring clamp consisting of said external clamping elements.

With these arrangements, one can reliably, accurately, and reproducibly prepare the organ wall to form a tubular portion extending from its opening, said portion having a geometry calibrated for an end-to-end or abutting connection to a hollow tube. For this purpose, the external clamping elements placed on the outside of the tubular portion of the wall in the radial direction advantageously form an anchoring clamp that can be used to reconnect the hollow tube.

The invention has a particularly advantageous application in the field of urology, in particular for reconstructing a patient's bladder wall and forming a tubular portion of predefined geometry on the wall at an opening created for example as a result of a prostatectomy, in preparation for an anastomosis with the patient's urethra.

However, it is conceivable to use the tubular portion formed by means of the system according to the invention to connect an inorganic hollow tube to the organ wall, for example a tube that is part of a prosthesis, particularly a vascular prosthesis.

According to the invention, the supporting body has at least one cylindrical or substantially cylindrical portion, and the biasing elements are used to move the external clamping elements towards the supporting body, said external clamping elements pulling the wall towards the supporting body by bearing on portions of an annular surface extending around the wall opening. The wall is then progressively deformed around its opening until it presses against the cylindrical portion of the supporting body and adopts a substantially tubular shape as it is sandwiched between said supporting body and the external clamping elements.

The external clamping elements may advantageously have at least one gripping and/or attachment hook to establish the gripping and/or attachment of said external clamping elements to the portions of the annular surface of the wall extending around the opening.

Advantageously, the system comprises an applicator for placing the supporting body through the opening formed in the wall and for fastening said supporting body to said wall. This applicator is adapted for removable connection to the proximal portion of the supporting body and for at least partially controlling the biasing elements, in particular from outside the patient's body.

In an advantageous arrangement of the invention, the biasing elements comprise connecting elements, each connecting element being adapted to connect an external clamping element to the supporting body, and a device for exerting traction on the connecting elements, said connecting elements each having a first end connected to said traction device and a second end adapted to be retained on an associated external clamping element, and actuation of said traction device tightening and juxtaposing said external clamping elements on said supporting body. The connecting elements are used to connect the external clamping elements and the supporting body by passing through the wall. These connecting elements are preferably flexible. They can advantageously be in the form of thread, for example of a bioresorbable material.

The traction device can advantageously be housed inside the supporting body and controlled from the applicator which places and stabilizes the supporting body. Alternatively, the traction device can be carried and controlled by the applicator.

Advantageously, it is possible for the traction device to be provided with a force-limiting system to limit the squeezing of the wall against the supporting body by the external clamping elements. This minimizes the risk of injury to organ tissue due to excessive tightening of the connecting elements.

Preferably, a return prevention device can be provided between the connecting elements and the supporting body, said device being adapted to prevent the external clamping elements from loosening on the supporting body when actuation of the traction device ceases.

According to another advantageous arrangement of the invention, the system further comprises a feedthrough and anchoring device adapted to move the second end of each connecting element between a retracted configuration where said second end is located at a radial distance from the longitudinal axis that is less than the radius of the opening, and an anchored configuration where said second end is retained on an associated external clamping element after passing through the wall. Thus, the second ends of the connecting elements in the retracted configuration can be inserted through the opening, and then moved to the anchored configuration such that they pass through the wall and are anchored in the external clamping elements placed on the other side of the wall.

Preferably, the feedthrough and anchoring device comprises support members for releasably supporting the second ends of the connecting elements.

Advantageously, the second ends of the connecting elements are provided with an anchoring element adapted to engage with a corresponding anchoring portion provided on the external clamping elements in order to retain said second ends on said external clamping elements when they are in the anchored configuration after passing through the wall.

The support members of the feedthrough and anchoring device and/or the anchoring elements equipping the second ends of the connecting elements may advantageously have a tip able to pierce the wall when said second ends move from their retracted configuration to their anchored configuration.

Preferably, in the retracted configuration, the second end of each connecting element is located at a radial distance from the longitudinal axis that is less than or substantially equal to the outer radius of the supporting body.

Advantageously, the feedthrough and anchoring device is integrated into the supporting body, and extends into the distal portion of said body intended for introduction through the wall opening.

Thus, in the retracted configuration, the connecting elements and the feedthrough and anchoring device can be accommodated inside the supporting body, which minimizes the risk of its catching on and damaging the tissue or affecting the function of the system according to the invention when it is inserted into the patient's body.

According to a preferred embodiment of the invention, this feedthrough and anchoring device can be controlled by the applicator, for example by means of a cable or a coupling rod.

According to a particular embodiment of the invention, the system further comprises a deployment mechanism adapted to move said external clamping elements at least between a retracted configuration where they are folded in the direction of the longitudinal axis, and a deployed configuration where they are away from the longitudinal axis and flare outwards substantially perpendicularly to it, and the deployment mechanism comprises support members on which the external clamping elements are releasably supported.

The deployment mechanism is adapted to move the external clamping elements between the deployed configuration and a collapsed configuration either simultaneously or sequentially. In the case of a sequential deployment of the external clamping elements by the deployment mechanism, the external clamping elements can be moved individually, one after the other, in a predetermined sequence, for example one after the other circumferentially or in a more dispersed manner. It is also conceivable to have sequential deployment in subsets of external clamping elements, the external clamping elements that are part of each subset being regularly distributed circumferentially. Thus, in the case of six external clamping elements, it is possible for example to provide two successive deployment sequences, namely a first deployment sequence in which three clamping elements spaced 120° apart circumferentially are deployed, followed by a second deployment sequence in which three clamping elements spaced 120° apart circumferentially and located between the external clamping elements deployed in the first sequence, are deployed. Again in the case of six external clamping elements, three successive deployment sequences could be provided for example, by pairs of diametrically opposed external clamping elements.

It can thus advantageously be arranged so that, when the second ends of the connecting elements and the external clamping elements are in the retracted configuration, one can insert from outside the patient's body, particularly by means of the applicator, the assembly consisting of the supporting body, the deployment mechanism with said external clamping elements, and the biasing elements at least partially housed inside the supporting body, through a through-hole formed in a trocar placed on an external body wall of the patient, for example the abdominal wall.

Advantageously, the deployment mechanism is provided on the end of a distal portion of the applicator, adapted for connection to the supporting body, said mechanism preferably being controlled by means of said applicator.

According to a particularly advantageous arrangement, the applicator, and in particular the deployment mechanism arranged on the distal end of said applicator, comprises a stop member adapted to cooperate with the wall to limit the insertion of the supporting body through the opening. Alternatively, the stop member could be provided directly on the supporting body.

In a first embodiment, the feedthrough and anchoring device is adapted to move the second end of each connecting element between the retracted configuration and the anchored configuration when the external clamping elements are in the deployed configuration.

In a second embodiment, the deployment mechanism is further adapted to move the external clamping elements between the deployed configuration and a collapsed configuration where said external clamping elements are folded in the direction of the longitudinal axis on the distal end side of the supporting body, and said deployment mechanism is part of the biasing elements.

In this embodiment, the external clamping elements in the deployed configuration can be moved to a collapsed configuration by pulling and progressively deforming the wall so that it is pressed against the supporting body and adopts a substantially tubular shape as it is sandwiched between said supporting body and the external clamping elements.

Similarly to the movement between the retracted configuration and the deployed configuration of the external clamping elements, the deployment mechanism is adapted to move the external clamping elements either simultaneously or sequentially between the deployed configuration and the collapsed configuration.

In this second embodiment, the feedthrough and anchoring device is adapted to move the second end of each connecting element between the retracted configuration and the anchored configuration when the external clamping elements are in the collapsed configuration.

According to a particularly advantageous arrangement of the present invention, the supporting body releasably supports internal clamping elements angularly aligned circumferentially with the external clamping elements, and the biasing elements are arranged to tighten and juxtapose said external clamping elements on said internal clamping elements. These internal clamping elements, when they are retained on the supporting body, define a substantially cylindrical portion therewith, which the wall is pressed against via the biasing elements, causing it to adopt a substantially tubular shape. When the internal clamping elements are separated from the supporting body, the wall is sandwiched between the external and internal clamping elements, and the supporting body can be withdrawn from the opening and removed from the patient's body, particularly by means of the applicator.

Advantageously, the system further comprises an ejection device adapted to move the internal clamping elements between a retracted configuration where they are at least partially housed inside the supporting body, and an ejected configuration where they are removed from said supporting body. Preferably, the ejection device is integrated with the supporting body. It may be controlled by means of the applicator.

Preferably, each connecting element comprises a portion associated with a respective internal clamping element, the system further comprising a cutting device adapted for cutting the connecting elements between the internal clamping elements and the first ends of said connecting elements. Preferably, the cutting device is integrated with the supporting body. It may be controlled by means of the applicator.

The internal clamping elements may advantageously be sized so that, after removal of the supporting body, they are held together in a circular configuration by their mutual contact with each other.

Additionally or alternatively, a retaining element may be provided that is able to retain the internal clamping elements and/or external clamping elements in a circular configuration after removal of the supporting body.

Through these arrangements, the hole created in the wall can be prepared so as to form a tubular portion on the wall which can be connected to an organic or inorganic hollow tube.

Still according to this first aspect, the invention also relates to a method for preparing a created opening on an organic wall, wherein:
a supporting body defining a longitudinal axis and having a proximal portion and a distal portion is provided;
external clamping elements are also provided;
the distal portion of the supporting body is introduced through the opening;
the external clamping elements are deployed so as to flare outward relative to the supporting body, said external clamping elements being distributed circumferentially and covering, around said opening, portions of an annular surface of said organic wall;
the external clamping elements are urged so as to tighten and juxtapose said external clamping elements on said supporting body in order to progressively deform said annular surface so as to, on the one hand, shape on said organic wall a tubular portion extending along the longitudinal axis, and on the other hand, form an anchoring clamp consisting of external clamping elements.

According to an advantageous arrangement, in the method for preparing a created opening on an organic wall according to the invention, with the supporting body releasably supporting internal clamping elements angularly aligned circumferentially with the external clamping elements, the external clamping elements are urged so as to tighten and juxtapose said external clamping elements on said internal clamping elements.

Preferably, in the method for preparing a created opening on an organic wall according to the invention, the internal clamping elements are moved between a retracted configuration where they are at least partially housed inside the supporting body, and an ejected configuration where they are withdrawn from said supporting body.

Again preferably, in the method for preparing a created opening on an organic wall according to the invention, with each connecting element comprising a portion associated with an associated internal clamping element, the connecting elements are cut between the internal clamping elements and the first ends of said connecting elements.

Another aim of the invention is to propose a system for performing anastomosis which provides a reliably reproducible and minimally invasive end-to-end connection of a tubular portion, formed by means of the system described above at an opening created in an organic wall, and an organic hollow tube.

Thus, according to a second aspect of the invention, a system is provided for performing an anastomosis between an organic wall having an opening and an organic hollow tube, said system comprising:
a system for preparing the opening as described above, and
a device for connecting said tube and the tubular portion shaped on the wall, said device being arranged at least partially within a proximal portion of the supporting body adapted for insertion into the tube and comprising:
   suture elements adapted for connecting the internal and external clamping elements and each having a first end connected to a tightening device and a second end adapted to be retained on an associated external clamping element; and,
   a suturing device adapted for moving the second end of each suture element between a retracted configuration where said second end is positioned at a radial distance from the longitudinal axis that is less than the inside radius of said tube, and an anchored configuration where said second end is retained on an associated external clamping element after passing through the tube from the inside to the outside.

With these arrangements, an end-to-end connection is created between the organic hollow tube and the tubular portion formed on the organic wall, so as to establish direct contact between their respective mucosa, which is particularly advantageous in terms of healing and complete and reliable restoration of the communication between the two organs, particularly between the patient's bladder and urethra.

The suture elements connect the external clamping elements and the supporting body by passing through the tube. These suture elements are preferably flexible. They can advantageously be in the form of thread, made for example of a bioresorbable material.

Advantageously, the system further comprises a control probe for the device for connecting the organic hollow tube and the tubular portion formed on the wall. This probe is adapted to be introduced into the hollow tube, and to be removably connected to the proximal portion of the supporting body in order to at least partially control said device, especially from outside the patient's body.

The tightening device can advantageously be housed inside the supporting body and controlled from the control probe. Alternatively, the tightening device can be carried and controlled by the probe.

Advantageously, it can be arranged so that the tightening device is provided with a force-limiting system to limit the clamping force of the anastomosis. This minimizes the risk of injury to body tissues due to excessive tightening of the suture elements which can lead to stenosis in the anastomosis area.

Preferably, a return prevention device can be provided between the suture elements and the internal clamping elements, said device being adapted to prevent the suture elements from loosening when actuation of the tightening device ceases.

In preferred embodiments of the invention, one or more of the following arrangements may be used:
each suture element comprises a portion associated with a respective internal clamping element, the system further comprising a cutting device adapted for cutting the suture elements between the internal clamping elements and the first ends of said suture elements. Advantageously, this cutting device is the same as the cutting device described above in relation to the preparation system which cuts the connecting elements;
the suturing device is further adapted to move the second end of the suture elements between the retracted configuration and the anchored configuration by passing through an intermediate deployed configuration where said second end is located opposite a corresponding anchoring portion provided on the associated external clamping element;
the suturing device comprises resiliently flexible elements, angularly aligned circumferentially with the internal and external clamping elements and axially movable within the supporting body, releasably supporting the second end of an associated suture element and adapted to cooperate with associated ramps provided in the supporting body so as to move said second end between the retracted configuration and the anchored configuration;
the second ends of the suture elements are provided with an anchoring element adapted to engage with a corresponding anchoring portion provided on the external clamping elements in order to retain said second ends on said external clamping elements when they are in the anchored configuration, after passing through the tube;
the flexible elements and/or anchoring elements equipping the second ends of the suture elements have a tip adapted to pierce the tube when said second ends move from their retracted configuration to their anchored configuration;
the ramps are movable axially inside the supporting body between first and second axial positions, the transition of said ramps from the first axial position to the second axial position causing the second end of the suture elements to move from the intermediate configuration to the anchored configuration due to elastic return of the flexible elements.

Still according to this second aspect, the invention also relates to a method for performing an anastomosis between an organic wall having an opening and an organic hollow tube, wherein:
the opening created on the wall is prepared in accordance with the method according to the first aspect of the invention;
the proximal portion of the supporting body is introduced at least partially into the tube;
suture elements are provided, adapted for connecting the internal and external clamping elements and each having a first end connected to a tightening device and a second end adapted to be retained on an associated external clamping element; and,
the second end of each suture element is moved between a retracted configuration where said second end is located at a radial distance from the longitudinal axis that is less than the inside radius of said tube, and an anchored configuration where said second end is retained on an associated external clamping element after passing through the associated tube from the inside to the outside.

In an advantageous arrangement, in the method for performing an anastomosis between an organic wall having an opening and an organic hollow tube according to the invention, each suture element comprising a portion associated with a respective internal clamping element, the suture elements are cut between the internal clamping elements and the first ends of said suture elements.

With these arrangements, the anastomosis performed using the system according to the invention is maintained, after withdrawal of the supporting body, by means of suture elements connecting the internal clamping elements to the external clamping elements by passing through the organic hollow tube from the inside to the outside in the radial direction and straddling the anastomosis area, said internal and external clamping elements being held together on the wall by means of connecting elements passing through the wall, in order to define a tubular portion thereon. In this manner, the respective edges of the organic hollow tube and the tubular portion formed on the organic wall are rendered contiguous, as tubular extensions of one another, forming an interface with direct contact between their respective mucosa.

It is particularly advantageous if the external clamping elements, the internal clamping elements, the connecting elements, and/or the suture elements are made of a bioresorbable material, meaning a material which can be resorbed by the physico-chemical activity of living tissue in contact with the material. For example, the component material of these elements can be a bioresorbable polymer such as polylactic acid (PLA, PLLA), polyglycolic acid (PGA), or polydioxanone (PDO).

Thus, the elements left within the patient's body after preparation of the created opening on the organic wall, using the system according to the first aspect of the invention, or after anastomosis between said wall and the organic hollow tube performed by means of the system according to the second aspect of the invention, will be resorbed through contact with the body tissue and the surrounding fluids, and it will not be necessary to schedule a subsequent procedure to remove these elements.

Finally, the invention also provides for the case where a tubular portion is formed on the organic wall by a means other than the preparation system described above.

A second object of the invention therefore relates to a system for performing an anastomosis between an organic wall having an opening and an organic hollow tube, said system comprising:
a supporting body defining a longitudinal axis and adapted for insertion inside the tube and through the opening;
at least one external clamping element defining with said body a tubular portion on the wall and forming an anchoring clamp;
a device for connecting said tube and said tubular portion defined on said wall, said device comprising:
   at least one suture element adapted for connecting the external clamping element to the supporting body and having a first end connected to a tightening device and a second end, said second end being adapted to be retained on the external clamping element; and
   a suturing device adapted for moving the second end of the suture element between a retracted configuration where said second end is located at a radial distance from the longitudinal axis that is less than the inside radius of said tube, and an anchored configuration where said second end is retained on the external clamping element after passing through the tube from the inside to the outside.

With these arrangements, an end-to-end connection is created between the organic hollow tube and the tubular portion defined on the organic wall, establishing direct contact between their respective mucosa, which is particularly advantageous in terms of healing and the complete and reliable restoration of communication between the two organs, particularly between the patient's bladder and urethra.

The suture element connects the external clamping element and the supporting body by passing through the tube. This suture element is preferably flexible. It may advantageously be in the form of thread, made for example of a bioresorbable material.

In preferred embodiments of the invention, one or more of the following arrangements may be used:
the suturing device is further adapted for moving the second end of the suture elements between the retracted configuration and the anchored configuration by passing through an intermediate deployed configuration where said second end is positioned facing a corresponding anchoring portion provided on the associated external clamping element;
the suturing device comprises at least one resiliently flexible element that is movable axially within the supporting body, releasably supporting the second end of the suture element and adapted to cooperate with a ramp provided in the supporting body so as to move said second end between the retracted configuration and the anchored configuration;
the ramp is movable axially within the supporting body between first and second axial positions, the transition of said ramp from the first axial position to the second axial position causing the second end of the suture elements to move from the intermediate configuration to the anchored configuration by elastic return of the flexible element;
the supporting body releasably supports at least one internal clamping element, and the suture element is adapted for connecting the external clamping element and said internal clamping element.
the system further comprises an ejection device adapted for moving the internal clamping element between a retracted configuration where it is at least partially housed inside the supporting body, and an ejected configuration where it is removed from said supporting body.
each suture element comprises a portion associated with a respective internal clamping element, the system further comprising a cutting device adapted for cutting the suture elements between the internal clamping elements and the first ends of said suture elements;
the system further comprises at least one connecting element adapted for connecting the external clamping element to the supporting body to form the anchoring clamp;
the system further comprises a device for exerting traction on the connecting element, and said connecting element has a first end connected to the traction device and a second end adapted to be retained on an external clamping element;
the system further comprises a feedthrough and anchoring device adapted for moving the second end of the connecting element between a retracted configuration where said second end is positioned at a radial distance from the longitudinal axis that is less than the radius of the opening and an anchored configuration where said second end is retained on an associated external clamping element after having passed through the wall.

Advantageously, this system may comprise a single control element adapted for controlling the various devices during a single operative phase of anastomosis. This control element consolidates, for example, all control functions of the applicator and probe which are carried out as part of the first object of the invention.

Still according to this second object, the invention also relates to a method for performing an anastomosis between an organic wall having an opening and an organic hollow tube, wherein:
a supporting body defining a longitudinal axis is provided;
at least one external clamping element is also provided, defining with said body a tubular portion on the wall and forming an anchoring clamp;
the supporting body is inserted into the tube and through the opening;
at least one suture element is provided, adapted for connecting the external clamping element to the supporting body and having a first end connected to a tightening device and a second end, said second end being adapted to be retained on the external clamping element; and
the second end of the suture element is moved between a retracted configuration where said second end is positioned at a radial distance from the longitudinal axis that is less than the inside radius of said tube, and an anchored configuration where said second end is retained on the external clamping element after having passed through the tube from the inside to the outside.

Other features and advantages of the invention will be apparent from the following description of one of its embodiments, given by way of non-limiting example, with reference to the accompanying drawings.

### BRIEF DESCRIPTION OF THE DRAWINGS

In the drawings:

FIG. 1 represents a schematic sectional view of an organic wall having an opening, in this case the bladder wall of a patient who has undergone a prostatectomy, and an organic hollow tube, in this case the patient's urethra, between which an anastomosis is to be performed;

FIG. 2 is a schematic sectional view of the bladder wall on which a tubular portion has been formed near the opening in preparation for an end-to-end anastomosis with the patient's urethra;

FIG. 3 is a perspective view of a complete anastomosis system comprising a system for preparing the opening in the bladder wall according to a first embodiment of the invention;

FIG. 4 represents an exploded perspective view of the supporting body according to the first embodiment of the invention;

FIG. 5 shows a perspective view of the system for preparing the opening on the bladder wall according to the first embodiment of the invention, prior to insertion through the opening;

FIGS. 5a to 5f are longitudinal half section views along line VV of FIG. 5, illustrating the successive phases of preparing the opening formed on the wall by means of the system according to the first embodiment of the invention;

FIG. 6 is a perspective view of the bladder wall with a tubular portion formed at its opening, ready for reconnection to the patient's urethra;

FIG. 7 is an exploded perspective view of the supporting body according to a second embodiment of the invention;

FIG. 8 is a view similar to FIG. 5, illustrating the system for preparing the opening in the organic wall according to the second embodiment of the invention, prior to insertion through the opening;

FIGS. 8a-8f are longitudinal half section views along line VIII-VIII of FIG. 8, illustrating the various successive phases of preparing the opening formed on the wall by means of the system according to the second embodiment of the invention;

FIG. 9 is a schematic view of a partial sectional elevation showing a first step of using the anastomosis system of the invention, associated with a patient's urethra that is to be anastomosed or reconnected to the tubular portion formed on the bladder wall by means of the preparation system according to the first embodiment of the invention;

FIGS. 10a to 10c are longitudinal half section views along line IX-IX of FIG. 9, illustrating the successive phases of an anastomosis between the patient's bladder and urethra using the system according to invention, the opening of the bladder wall having been prepared by means of the preparation system according to the first embodiment of the invention;

FIG. 11 is a longitudinal half section view along line IX-IX of FIG. 9 similar to those of FIGS. 10a to 10c, illustrating the step of ejecting the internal clamping elements after anastomosis of the tubular portion, formed on the bladder by means of the preparation system according to the first embodiment of the invention, and the patient's urethra, and of cutting the connecting elements and suture elements, enabling the anastomosis system according the invention to be withdrawn from the patient's body;

FIG. 12 is a longitudinal half section view of the wall of the bladder and urethra of the patient after anastomosis using the system according to the invention, and withdrawal of said system from the patient's body.

### DETAILED DESCRIPTION OF THE DISCLOSURE

In the different figures, the same references denote identical or similar elements.

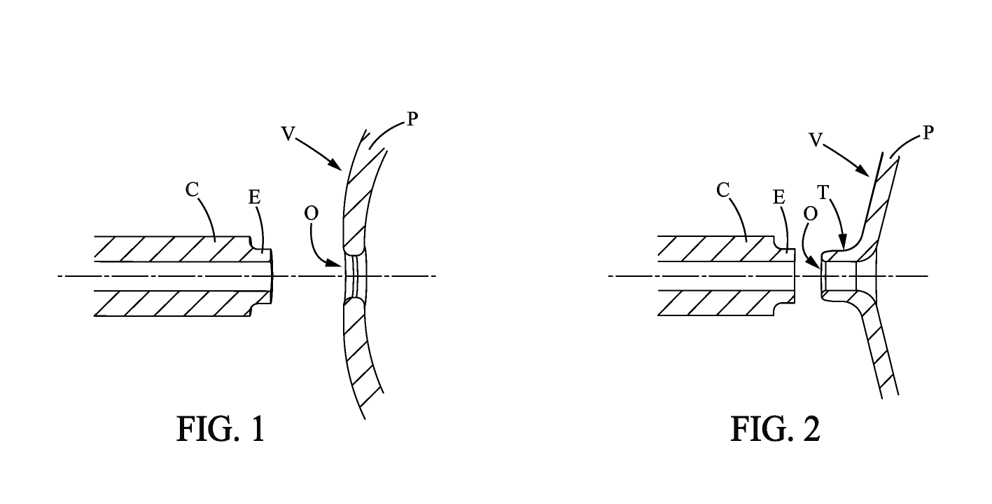

FIG. 1 illustrates an organic wall P having an opening O, and an organic hollow tube C having an open end E.

In the following description, it is assumed that this wall P is part of a bladder V and that the opening O in it was created during a prostatectomy. Furthermore, the organic hollow tube C considered in the context of the present description is the patient's urethra, of which the open end E, resulting from a prior resection of the urethra in its membranal area that was performed during the prostatectomy, needs to be reconnected to the wall P of the bladder V during an anastomosis operation in order to restore communication between the bladder and the terminal opening of the urethra opposite the urethral meatus, which is open at this end E.

FIG. 2 shows a sectional diagram of the wall P of the bladder V which has been deformed to shape a tubular portion T at the opening O, said tubular portion T being intended for end-to-end reconnection during an anastomosis operation at the end E of the urethra C that is opposite the urethral meatus.

A first aim of the invention is to propose a system SP, SP′ for preparing the wall P of the bladder V so as to form this tubular portion T at the opening O.

A second aim of the invention is to propose an anastomosis system SA for establishing the end-to-end connection of the tubular portion T, formed by means of said system SP, SP′ at the opening O created on the wall P of the bladder V, and the patient's urethra.

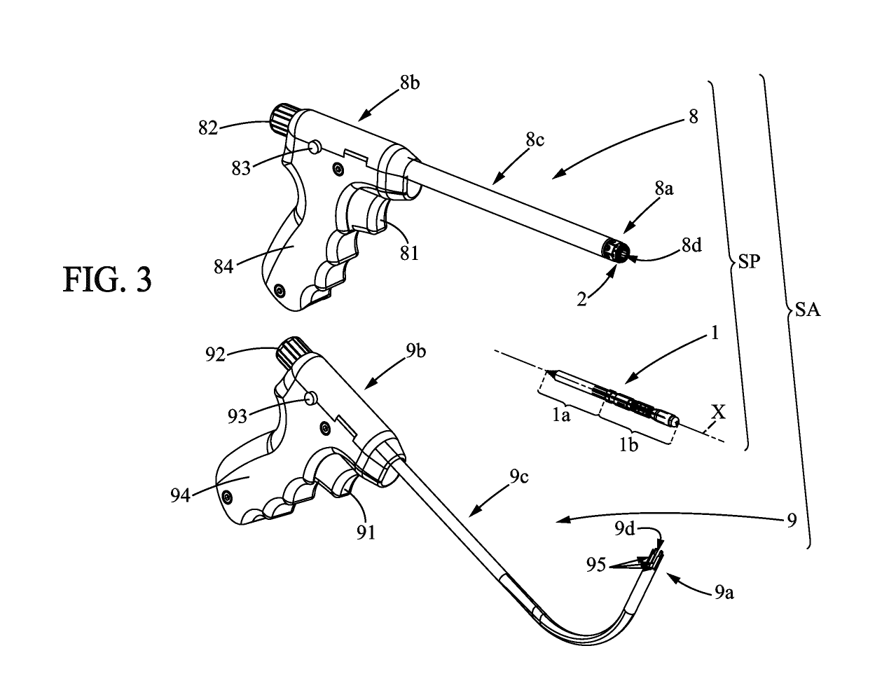

In FIG. 3, a complete system for performing an anastomosis SA between a patient's bladder and urethra is represented. The anastomosis system SA comprises a system SP for preparing the opening O of the wall P of the bladder V according to a first embodiment. This system SP comprises:
a supporting body 1 defining a longitudinal axis X and having a proximal portion 1a and a distal portion 1b, said distal portion 1b being adapted for insertion through the opening O, and
an applicator 8 having a proximal control portion 8b for manipulation by the surgeon, a distal portion 8a to which the proximal portion 1a of the supporting body 1 is intended to be connected during a first operating phase or tubular portion creation phase, and a central elongate portion 8c.

The anastomosis system SA further comprises a probe 9 having a proximal control portion 9b for manipulation by the surgeon, a distal portion 9a to which the proximal portion 1a of the supporting body 1 is intended to be connected during a second operating phase or anastomosis phase, and a central elongate portion 9c.

In the embodiment illustrated in FIG. 3, the distal portion 8a of the applicator 8 has a housing 8d for receiving the proximal portion 1a of the supporting body 1. The distal portion 8a of the applicator 8 is equipped with a deployment device 2 which will be described in more detail below.

As can also be seen in FIG. 3, the distal portion 9a of the probe 9 has a housing 9d for receiving the proximal portion 1a of the supporting body 1. The distal portion 9a has a plurality of slots 95 opening into the central housing 9d whose usefulness will be described below.

In a manner not detailed in the figures, in either of the illustrated embodiments of the preparation system SP, the distal portions 8a, 9 of the applicator 8 and probe 9 are shaped for assembly in a complementary manner to the proximal end of the supporting body 1, 1′, for the purposes of controlling the various devices provided on or within said supporting body 1, 1′. Various embodiments are possible for this, as long as the applicator 8 and probe 9 are able to transmit the control movements of these devices from their proximal portion 8b, 9b visible in FIG. 3. In the example shown, these proximal portions 8b, 9b are in the form of a manually actuated device comprising a handle 84, 94 and various control elements, such as a trigger 81, 91, a knob 82, 92 that can be rotated and moved in translation, or a locking button 83, 93.

Alternatively, it could be arranged so that the proximal portion of the applicator 8 and/or probe 9 is mechanically connected to an interface linked to a robotic arm, to allow control of the system(s) by a computer under surgeon supervision, particularly in a context of computer-assisted surgery performed remotely.

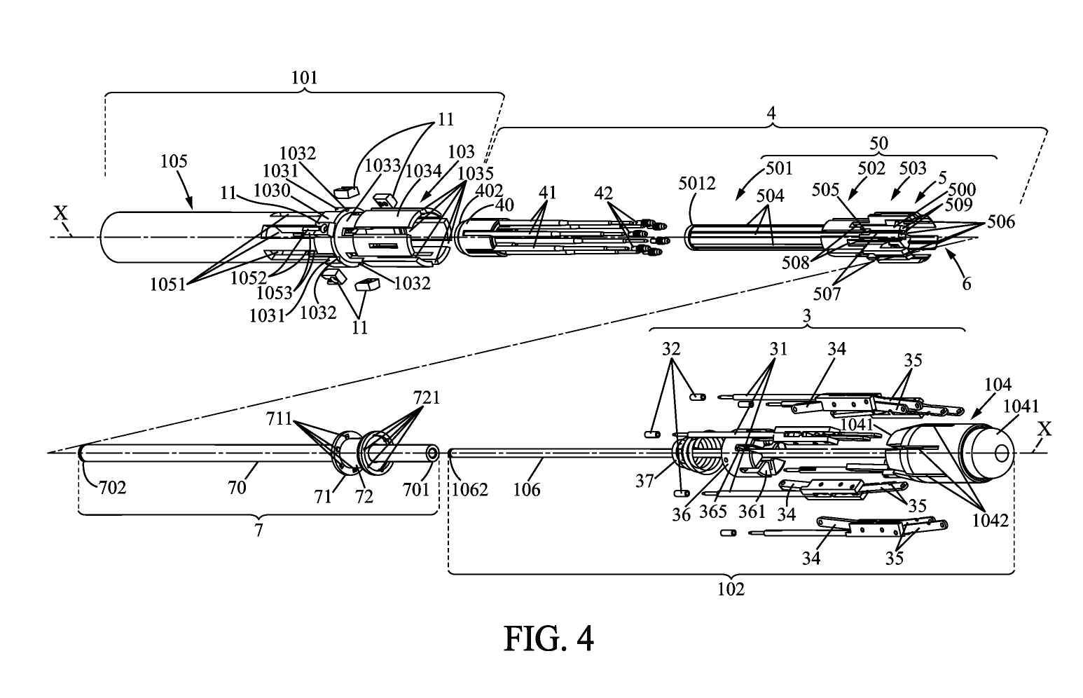

FIG. 4 illustrates an exploded perspective view of a supporting body 1 that is part of an anastomosis system SA comprising a preparation system SP according to a first embodiment of the invention.

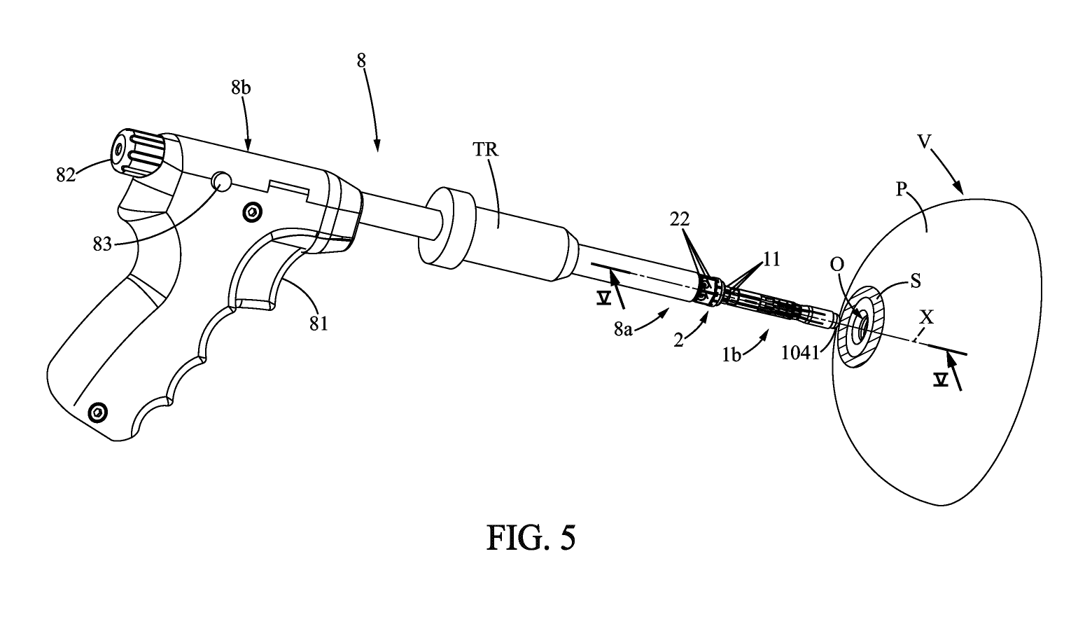

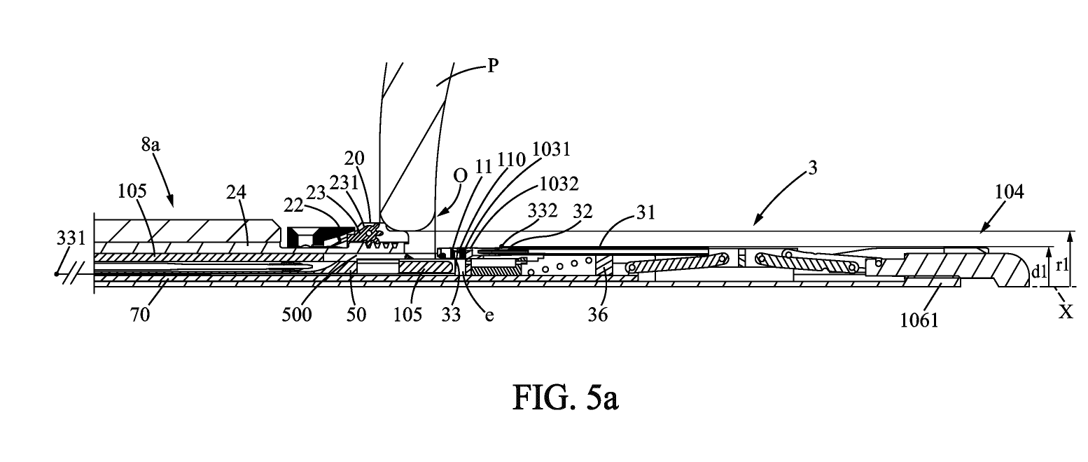

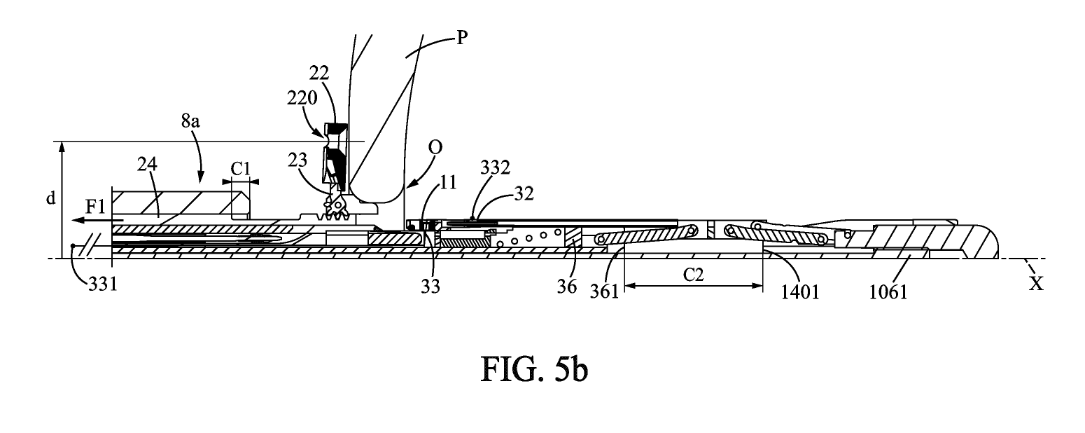

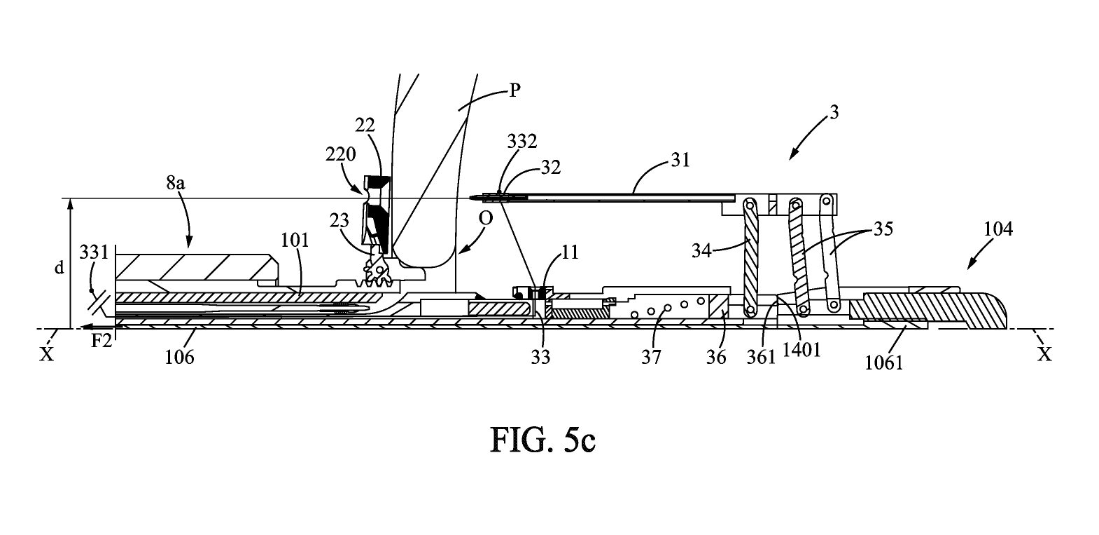

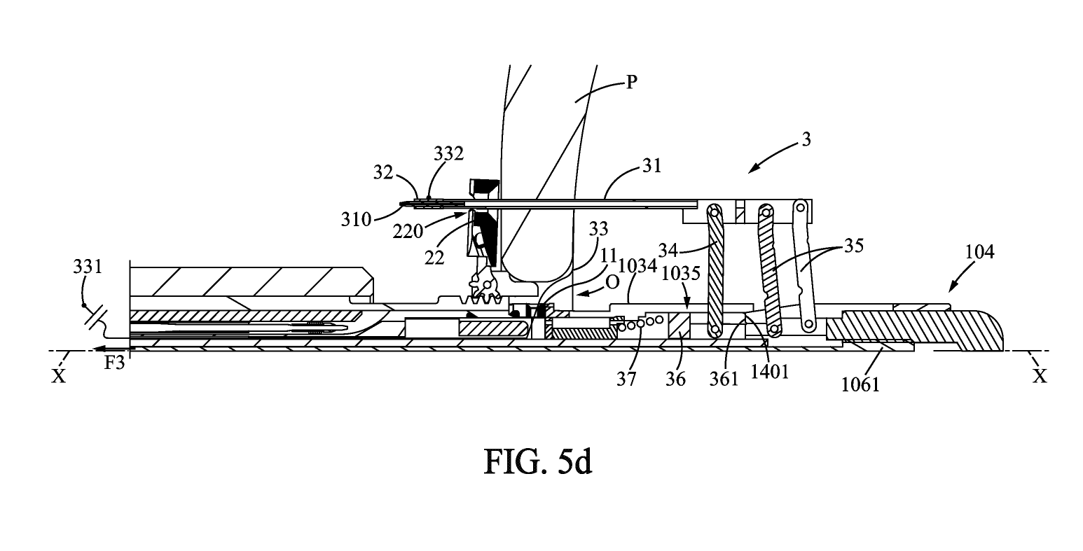

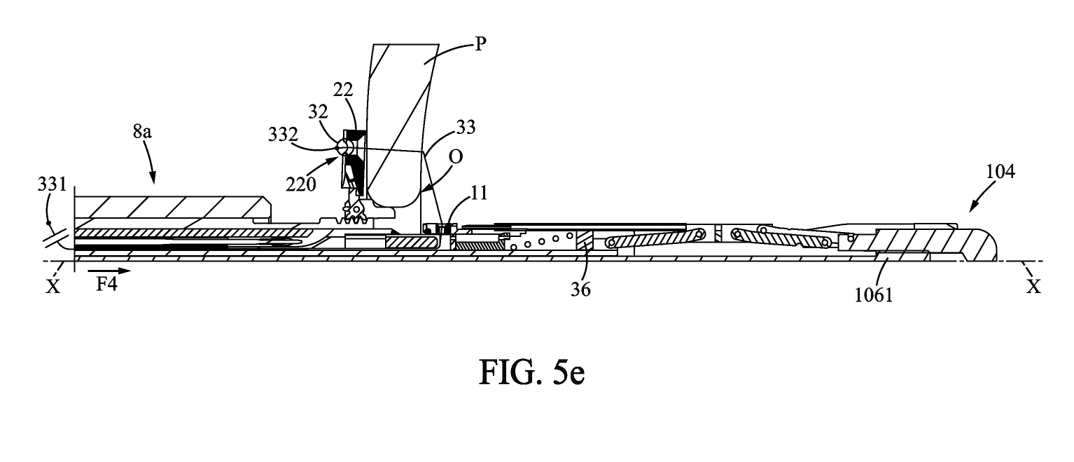

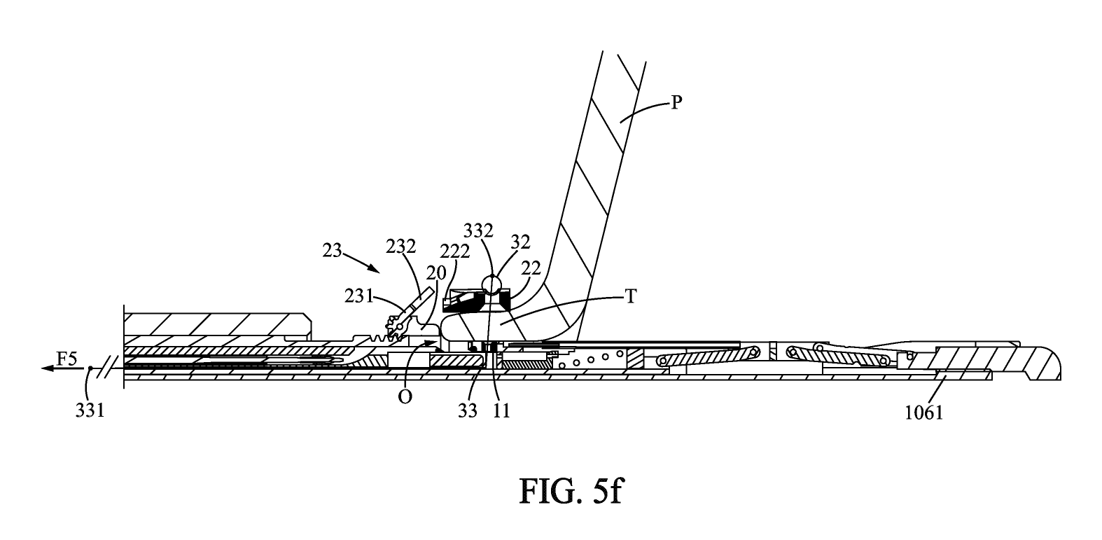

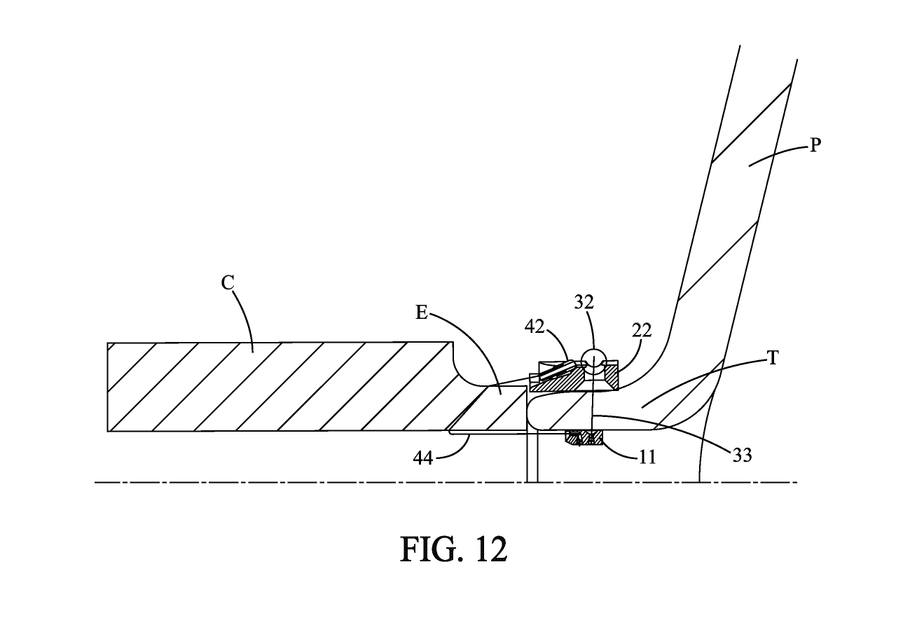

In this figure, the connecting elements 33 intended to pass through the wall P of the bladder V, and the suture elements 44 intended to pass through the urethra, have not been represented in order to improve clarity; these elements, which are in the form of threads made of bioresorbable materials, are particularly visible in FIGS. 5a to 5f, 10a to 10f, 11 and 12.

As can be seen in FIG. 4, the supporting body 1 is in two parts 101, 102 which are movable relative to each other along the longitudinal axis X.

The first part, which will be considered here as the fixed part, is in the form of a generally cylindrical member 101 extending along the longitudinal axis X and comprising two hollow cylindrical portions of different outside diameters: a proximal portion 105 having an outside diameter corresponding substantially to the inside diameter of the housings 8d, 9d provided in the distal portions 8a, 9a of the applicator 8 and probe 9, and a distal portion 103 having an outside diameter greater than that of the proximal portion 105.

The proximal portion 105 is axially open at its proximal end and has a plurality of radial slots 1051 along its periphery, here six in number, which connect its inside space to the outside. In the distal extension of these slots 1051, the proximal portion 105 has grooves 1052 on its outer surface which are not radially open to the inside space of the proximal portion 105 and in which the bottom has a longitudinal channel 1053 which opens axially into a corresponding slot 1051 on the proximal side, and into the inside space of the distal portion 103 on the distal side.

The distal portion 103 is axially open at its distal end and defines an inside space communicating with the inside space of the proximal portion 105. The grooves 1052 arranged in the proximal portion 105 extend axially into a proximal portion 1030 of the distal portion 103 and define a plurality of housings 1031 whose usefulness will be further described below. These grooves 1052 stop axially inside the proximal portion 1030 of the distal portion 103. The housings 1031 have a through-opening in their base that connects the inside space of the distal portion 103 with the outside, providing space e to allow the passage of each associated connecting thread 33 as is particularly visible in FIG. 5a.

The proximal portion 1030 of the distal portion 103 also has notches 1032 on its outer surface which lie in the axial extension of the housings 1031. The distal portion 103 has an annular groove 1033 and a distal portion 1034. The notches 1032 open axially inside the housing 1031 on the proximal side and inside the channel 1033 on the distal side.

The distal portion 103 also has a plurality of radial slots 1035 along its periphery, aligned circumferentially with slots 1051, radially connecting the inside space of the distal portion 103 with the outside, and opening axially at the distal end of the distal portion 103. The utility of these radial slots 1035 will be described in more detail below.

The proximal portion 1030 of the distal portion 103 has a generally cylindrical outer surface in which are arranged the housings 1031 provided for releasably receiving internal clamping elements 11, here in the form of inner plates made of a bioresorbable material, said plates 11 for example fitting tightly inside said housings 1031.

Still referring to FIG. 4, the second part of the supporting body 102, which will be considered here as the movable part, comprises a generally cylindrical member 104 having a bullet-nose distal end 1041, and a rod 106 extending along the longitudinal axis X of which the distal end 1061 is connected to the cylindrical member 104, for example by screwing or by force-fitting. In addition, the rod 106 has a proximal end 1062 adapted for insertion along the longitudinal axis X through the fixed part 101 of the supporting body 1, so as to project proximally beyond the proximal end of said fixed part 101, and intended to be connected to the applicator 8 in order to control the movement of the movable part 102 relative to the fixed part 101.

The cylindrical member 104 has a plurality of radial slots 1042 along its periphery, here six in number, connecting its inside space with the outside and aligned circumferentially with slots 1035 provided on the fixed part 101 of the supporting body 1.

A feedthrough and anchoring device 3 is arranged between the fixed part 101 and movable part 102 of the supporting body 1. This device 3 comprises a plurality of needles 31 of which the proximal end releasably supports anchoring elements 32 in the form of hollow cylinders which for example fit tightly on the distal end of the needle 31 with which it is associated, and to which are attached the second ends 332 of the connecting threads 33, the first end 331 of said threads 33 being connected to a traction device advantageously provided at the applicator. Advantageously, the hollow anchoring cylinders 32 are made of a bioresorbable material.

Each of the needles 31 is supported at a distal portion by a plurality of connecting rods, here a proximal rod 34 and two distal rods 35, hinged so as to define a deformable parallelogram mechanism. For this purpose, the distal rods 35 supporting each needle 31 are hinged on the movable part 102 of the supporting body 1, and on the distal portion of the needle 31 that they support; the proximal rod 34 is hinged on a slider 36 movable in translation along the rod 106 and on the distal portion of the needle 31 that it supports. A resilient element, which here is in the form of a coil spring 37 of frustoconical shape, is arranged so that it is compressed for at least a portion of the axial travel of the slider 36.

The slider 36 also has a plurality of radial through-slots 365 allowing a certain displacement of the proximal rods 34. The radial slots 1042 provided on the cylindrical member 104 of the movable part 102 of the supporting body 1 allow a certain displacement of the distal rods 35.

As can also be seen in FIG. 4, the supporting body 1 concentrically receives a hollow shaft 70 adapted to slip onto the rod 106 of the movable part 102 of the supporting body 1, and to be rotatable about the longitudinal axis X with respect to said rod 106. An annular ring 71 having a plurality of through-openings 711 is threaded onto the hollow shaft 70 so as to rotate in a floating manner. When the shaft 70 is placed inside the proximal part 101 of the supporting body 1, the ring 71 fits tightly over a corresponding bearing surface of said proximal part 101. Furthermore, the hollow shaft 70 is secured to a mandrel 72 having a plurality of through-holes and attachment holes 721 for suture threads 44 (not represented in FIG. 4).

The hollow shaft 70 has a distal end 701 and a proximal end 702 adapted for insertion along the longitudinal axis X through the fixed part 101 of the supporting body 1 so as to project in the proximal direction beyond the proximal end of said fixed part 101, and intended to be connected to the applicator 8 in order to control the shaft 70. The shaft 70 and mandrel 72 are part of a clamping device 7 whose operation will be further described below.

In the example represented, the slider 36 of the feedthrough and anchoring device 3 is mounted to be movable in translation along the longitudinal axis on a distal portion of the hollow shaft 70, and the compression spring 37 presses against a radial face of mandrel 72.

Still in FIG. 4, one can see that the supporting body 1 also concentrically receives a generally cylindrical tiered member 50, movable in translation along the longitudinal axis X relative to the fixed part 101 of the supporting body 1. This tiered member 50 is adapted to be threaded onto the hollow shaft 70 and has three cylindrical portions of different outside diameters: a proximal portion 501, a central portion 502, and a distal portion 503.

The proximal portion 501 has a proximal end 5012 adapted for insertion along the longitudinal axis X through the fixed part 101 of the supporting body 1, so as to project proximally beyond the proximal end of said fixed part 101, and intended for connection to the applicator 8 in order to control the tiered member 50.

A plurality of longitudinal channels 504, aligned circumferentially with the radial slots 1035 provided on the fixed part 101 of the supporting body 1, are provided on the outer surface of the proximal and central portions 501, 502. These channels 504 terminate at their distal end in a ramp 505 formed in the distal portion 503 and extending distally outwardly in the radial direction until it opens onto the outer surface of the distal portion 503 of the tiered member 50. The distal portion 503 has a plurality of radial through-slots 507 on its outer surface, aligned circumferentially with the channels 504 and having proximal ends which open axially inside the ramps 505.

Moreover, the distal portion 503 and central portion 502 have a plurality of radial through-slots 508 angularly offset to the channels 504 and defining on the distal portion 503 a plurality of wedges 500 extending as the axial extension of the channels 504. At their distal end, each wedge 500 has an ejection face 509 that is part of an ejection device 5 and is adapted to cooperate with the inner plates 11 in order to eject them from their housings 1031 in the radial direction, as will be further described below.

Furthermore, each of these ejection wedges 500 has two arms separated by a through-slot 507 and joined at their distal end by a cutting blade 506 that is part of an cutting device 6.

When the tiered member 50 is in place inside the fixed part 101 of the supporting body 1, the wedges 500 are inserted into slots 1051 provided in the proximal portion 105 of the fixed part 101 of the supporting body 1 and can move inside them by sliding along the bottom of grooves 1052.

Finally, the supporting body 1 concentrically receives a hollow sleeve 40 movable in translation along the longitudinal axis X relative to the fixed part 101 of the supporting body 1. The sleeve 40 has a proximal end 402 adapted for insertion along the longitudinal axis X through the fixed part 101 of the supporting body 1 so as to project proximally beyond the proximal end of said fixed part 101, and intended for connection to the applicator 8 for the purpose of controlling the displacement of the sleeve along the longitudinal axis.

The sleeve 40 is suitable for slipping onto the proximal portion 501 of the tiered member 50 and supports a plurality of resiliently flexible needles 41 suitable for inserting into the channels 504 of the tiered member 50 and for cooperating with the ramps 505 during operation.

The distal end of each needle releasably supports anchoring elements 42 in the form of hollow cylinders which, for example, fit tightly onto the distal end of the needle 41 with which it is associated, and to which are fixed the second ends 442 of the suture threads 44, the first end 441 of said threads 44 being connected to the tightening device 7, and more precisely at the attachment openings 721 of the mandrel 71.

In the embodiment illustrated in FIG. 4, the sleeve 40, needles 41, and tiered member 50 are part of a suturing device 4 which will be further described below. The ejection device 5 and cutting device 6 here are part of this suturing device 4.

The sleeve 40, needles 41, and tiered member 50 here are part of a suturing device 4 whose operation will be further described below.

Thus, in the embodiment illustrated in FIG. 4, all the elements carried by the supporting body 1, when in their initial configurations, fit within a cylinder whose diameter corresponds to the outside diameter of the distal portion 103 of the fixed part 101 of the supporting body 1. Preferably, this diameter is smaller than the diameter of the opening O present on the wall P so as to allow insertion of the distal portion 1a of the supporting body through the opening O.

We will now describe a first operative phase of preparing the opening O created on the bladder wall P, so as to form a substantially tubular portion thereon, making use of the preparation system SP according to the first embodiment of the invention.

For this purpose, as can be seen in FIG. 5, the proximal portion 1a of the supporting body 1 is inserted along its longitudinal axis X into the housing 8d defined in the proximal portion 8a of the applicator 8, and the distal portion 1b of the supporting body 1 extends distally from said proximal portion 8a of the applicator 8.

In this FIG. 5, one can see that in the first embodiment of the preparation system SP according to the invention, the distal portion 8a of the applicator supports a deployment mechanism 2 comprising a plurality of support members 23, particularly visible in FIGS. 5a to 5f, releasably supporting external clamping elements 22 to be placed facing the respective portions of an annular surface S of the wall P centered about the opening O.

The deployment mechanism 2 is able to move the external clamping elements 22 between a retracted configuration, shown in FIG. 5, where they are folded in the direction of the longitudinal axis X on the proximal end 1a side of the supporting body, and a deployed configuration where they are away from the longitudinal axis X and flare out substantially perpendicularly thereto.

The distal portion 8a of the applicator 8, fitted in this manner with the supporting body 1 in the initial configuration, and having external clamping elements 22 in the collapsed configuration, may advantageously be inserted through a trocar TR placed on the abdominal wall of the patient, and control of the various devices can be accomplished by means of the proximal portion 8b of said applicator 8 from outside the patient's body, typically during endoscopic surgery.

The applicator 8 fitted with the supporting body 1 is then manipulated by the surgeon so that the body 1 approaches the bladder V, until the distal portion 1a of said body is introduced into the opening O formed in the wall P, centering the longitudinal axis X within the opening O and engaging the distal end 1041 first. The bullet-nose shape of this end 1041 facilitates placement.

The external clamping elements 22, here six in number, are in the form of outer plates made of a bioresorbable material and are releasably supported on support members 23, for example by having complementary shapes.

The distal portion 8a of the applicator 8 and the proximal portion 1a of the supporting body 1 advantageously have indexing elements for fixing the supporting body 1 to the end of the applicator 8 so that the outer plates, supported by the applicator 8, are angularly aligned circumferentially with the inner plates 11 supported by the distal portion 1b of the supporting body 1.

Furthermore, the deployment mechanism 2 advantageously has a stop member 20 adapted to cooperate with the wall P to limit the insertion of the supporting body 1 through the opening O.

As can be seen in FIGS. 4 and 5, the assembly formed by the distal portion 8a of the applicator 8 and the supporting body 1 has rotational symmetry about the longitudinal axis X and can be divided into a plurality of identical basic modules uniformly distributed circumferentially. In the example shown, this assembly can be divided into six basic modules each extending over an angular range of about 60° and comprising an inner plate 11, an outer plate 22, a connecting thread 33 provided to connect the inner plate 11 and outer plate 22 after passing through the wall P, and a suture thread 44 provided to connect the inner plate 11 and outer plate 22 after passing through the urethra.

FIGS. 5a to 5f, as well as 10a to 10f, 11 and 12, illustrate just one of these modules. The reader will understand that what is visible in these figures applies to each of the other five modules.

Thus, FIGS. 5a to 5f show the various steps of preparing the opening O by means of the preparation system SP according to the first embodiment of the invention.

In FIG. 5a, the system SP is partially illustrated in its initial configuration and when placed at the opening O.

In this initial configuration of the preparation system SP, the deployment mechanism 2 is in the configuration with the outer plate 22 collapsed and supported by the support member 23, the feedthrough and anchoring device 3 is in the configuration where the second end 332 of the connecting thread 33 is retracted, this end 332 being at a radial distance d1 from the longitudinal axis X that is less than the radius r1 of the opening O, the inner plate 11 is received within its housing 1031 provided on the supporting body, and the ejection 5 and cutting 6 device, here formed by a wedge 500, is in the inactive configuration.

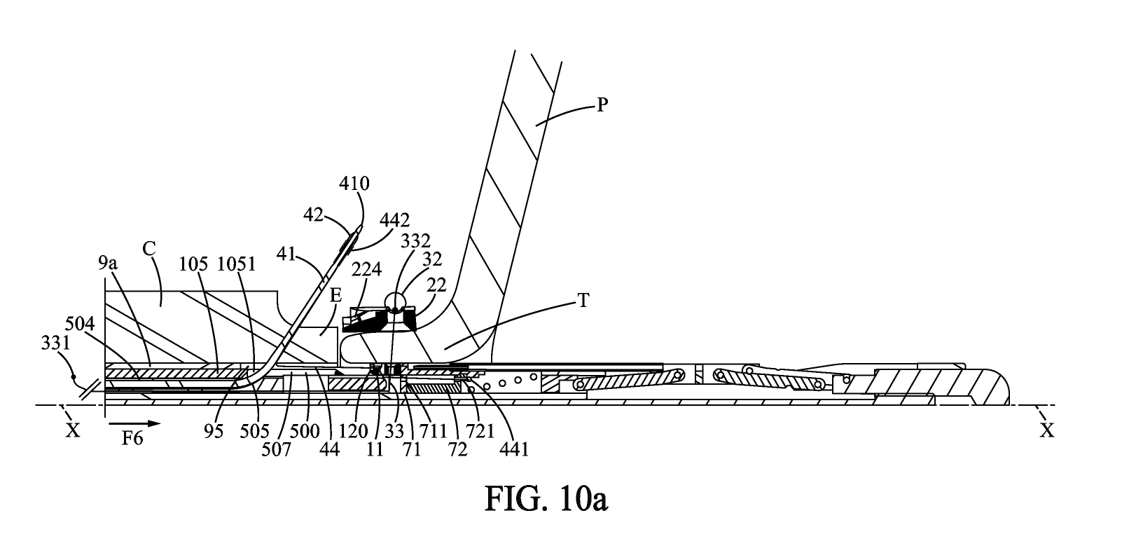

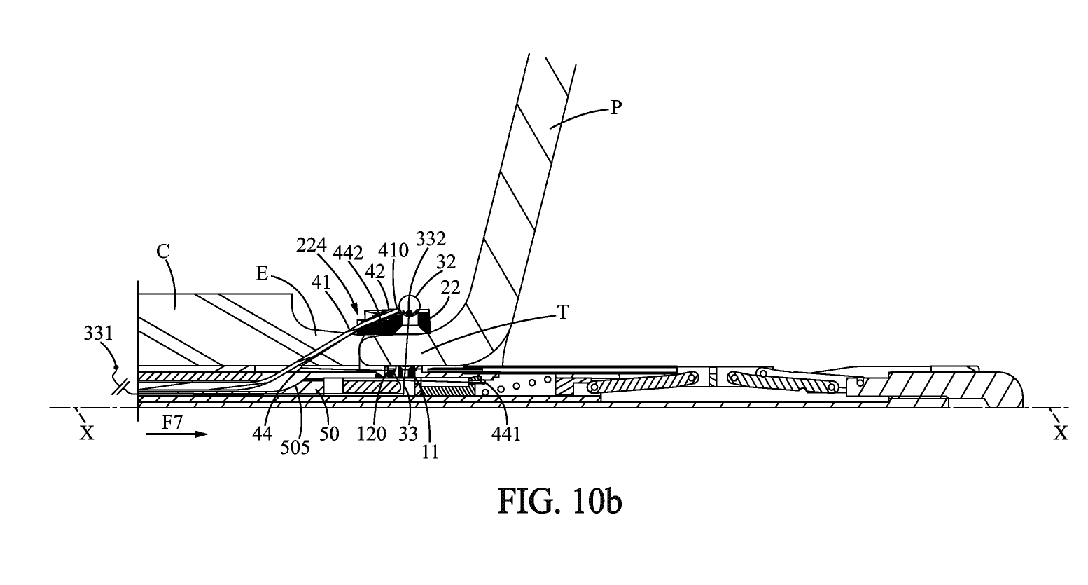

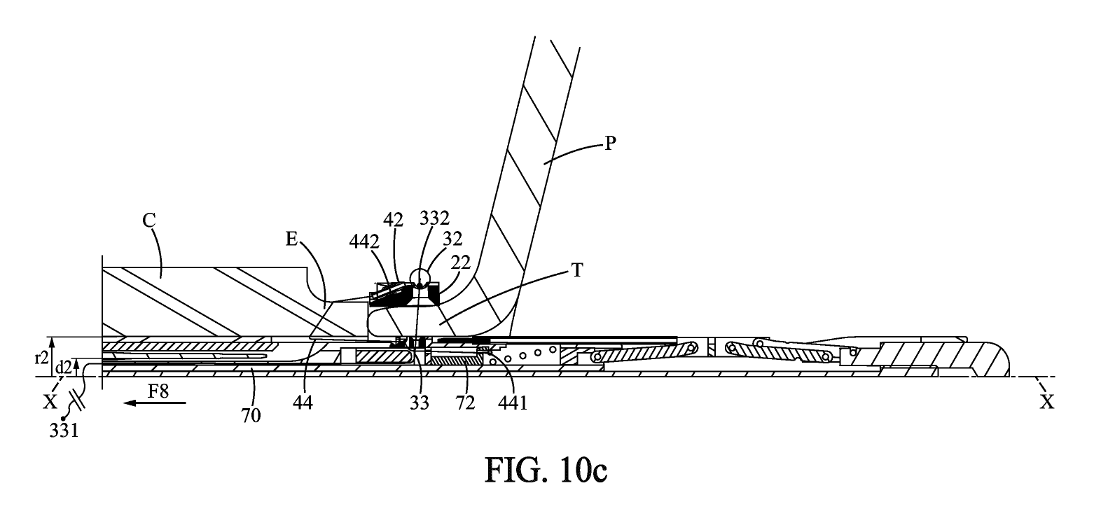

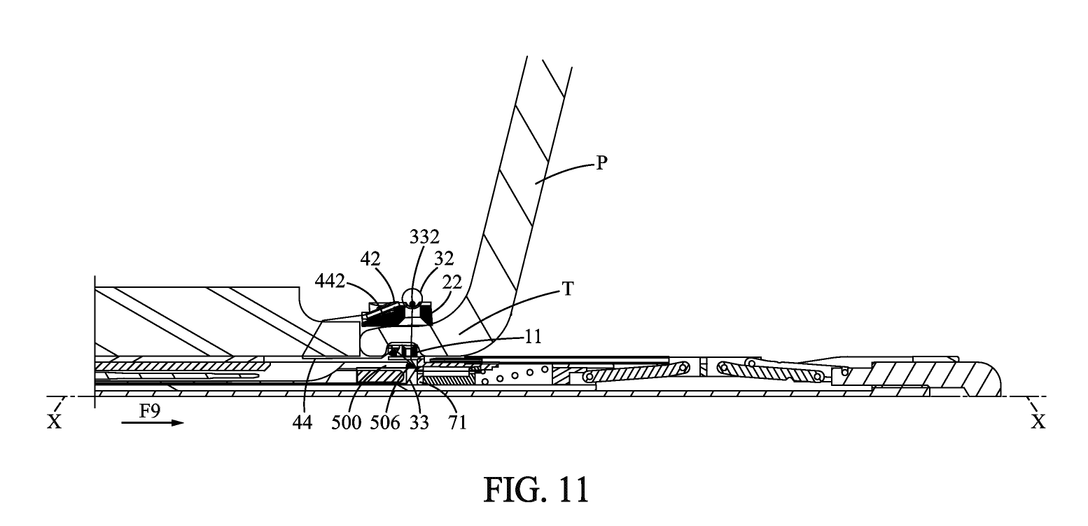

The suturing device 4 is also in the configuration where the second end 442 of the suture thread is retracted. This device will come into play during the second operative phase, or anastomosis phase, and its operation will be further described below in relation to FIGS. 10a to 10c and 11. For clarity, the suture thread 44 is not represented in FIGS. 5a to 5f.

Returning to FIG. 5a, one can see that in this first placement step, the supporting body 1 is inserted through the opening O until the stop member 20 comes in contact with the outer surface of the wall P, or the surface on the proximal side. This stop member 20 thus limits the insertion of the supporting body 1 through the opening O. In addition, the abutment of the stop member 20 against the wall P ensures correct axial positioning of the deployment mechanism 2 for the plate 2.

In a manner not represented in detail in the figures, the stop member 20 is integral to the distal portion 8a of the applicator 8.

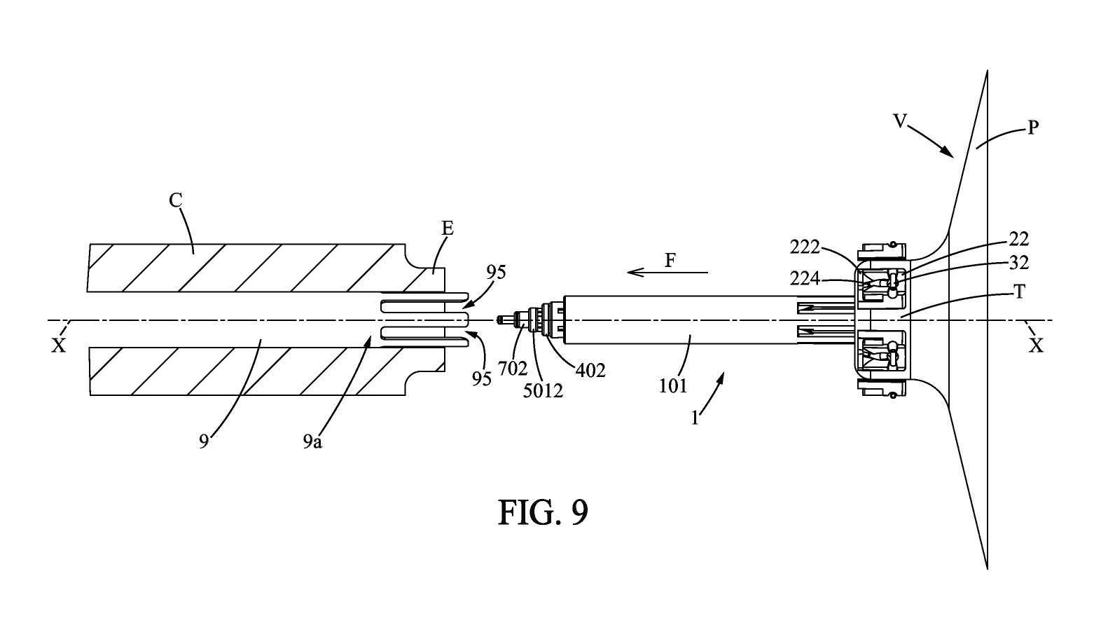

In FIG. 5a, one will note that the stop member 20 rotatably supports a support member 23 which is in the form of a generally Y-shaped fork having a lower arm 231 rotatably mounted on the stop member 20 and two upper arms 232 cooperating with a cavity 222 of complementary shape provided on the outer plate 22 to provide releasable support for said plate 22 on the support member 23 and visible in particular in FIG. 9. One of these upper arms 232 and part of the cavity 222 are visible in FIG. 5f.

The lower arm 231 has a plurality of peripheral gear teeth, adapted to cooperate with a rack provided on a distal portion of a sheath 24 mounted to be movable in translation inside the distal portion 8a of the applicator 8 and which can be controlled from the proximal portion 8b thereof. In the case of sequential deployment of the external clamping elements, the sheath 24 can be divided into as many parts as there are to be successive deployment sequences; each of these parts is intended to control the deployment of one or more clamping elements between the retracted configuration and the deployed configuration, and can be successively controlled from the proximal portion 8b of the applicator 8.

In the collapsed configuration of the deployment mechanism 2, the support 23 and the outer plate 22 extend substantially parallel to the longitudinal axis.

Also visible in FIG. 5a is the path of a connecting thread 33 within the supporting body 1. This thread 33 has a first end 331 adapted for connection to a traction device (not represented) built into the applicator 8. The thread 33 traverses the proximal portion 1a of the supporting body 1 within a space provided for this purpose, and extends substantially for the length of the hollow shaft 70. This thread 33 then emerges into a free space e provided in the supporting body 1 and located under the inner plate 11 received in its housing 1031. The connecting thread 33 then passes through the inner plate 11 by a through-hole 110, and changes direction again as it follows the outer surface of the plate 11; then it passes through the notch 1032 and is fixed by its second end 332 to an anchoring element 32 releasably supported by a needle 31.

Referring now to FIG. 5b, the sheath 24 has been moved by appropriate controls along an axial path C1 in the direction of arrow F1, which has caused the support member 23 to pivot due to the engagement of its teeth with the rack supported by the sheath, resulting in a deployed configuration of the outer plate 22 where it lies substantially perpendicular to the longitudinal axis X, facing a portion of the annular surface S of the wall P visible in FIG. 5.

In this deployed position, an anchoring portion 220 formed in the outer plate 22 and designed to retain the anchoring cylinder 32 to which the second end of the connecting thread 33 is fixed, is at a predetermined radial distance d from the longitudinal axis X.

Turning to FIG. 5c, traction in the direction of arrow F2 exerted on the rod 106 causes movement of the movable part 102 of the supporting body 1 with respect to the fixed part 101 of the body 1 along a first axial path, until the proximal end 1041 of the cylindrical member 104 and a distal stop surface 361 of the slider 36 come into contact. This axial path C2 of the movable part 102 relative to the fixed part 101, visible in FIG. 5b, causes deformation of the deformable-parallelogram mechanism consisting of the proximal rod 24 and distal rods 35. The feedthrough and anchoring device 3 is then in an intermediate deployed configuration where the second end 332 of the thread 33 is positioned facing the anchoring portion 220 of the outer plate 22 in the radial direction. In this position, the outer plate 22 and the second end 332 of the connecting thread 33 are facing each other radially, and are on opposite sides of the wall P axially.

By continuing the displacement of the rod 106 as indicated by arrow F3, visible in FIG. 5d, this causes movement of the movable part 102 relative to the fixed part 101 along a second axial path, driving the slider 36 via radial contact with the cylindrical member 104 and compressing the spring 37 to bear against a proximal face of the slider 36 and against a radial face of the mandrel 71. This drives the needle 31, by means of the connecting rods 34, 35, along the second axial path.

This second axial path can be defined in different ways, for example as the stroke required to move the spring 37 from its free state, where it is not subject to any stress, to a compressed state where its turns are touching. In any event, the second axial stroke is dimensioned so that the second end 332 of the connecting thread 33 and the anchoring cylinder 32 which supports it crosses through the wall P and outer plate 22 by passing through the anchoring portion 220. In the embodiment illustrated, the needle 31 has a tip 310 for piercing the wall P, on its proximal end.

By releasing the traction exerted on the rod 106, the relaxation of the spring causes the feedthrough and anchoring device 3 to return to its configuration shown in FIG. 5c. During this return movement, the needle 31 releases the anchoring cylinder 32 to which the second end 332 of the connecting thread 33 is attached, this cylinder 32 catching on the anchoring portion 220 of the outer plate 22. FIG. 9 shows a non-limiting example of cooperation between the anchoring cylinder 32 and the anchoring portion 220.

Next, by pushing the rod in the direction of arrow F4 visible in FIG. 5, the feedthrough and anchoring device 3 is returned to its retracted configuration.

It is now in the configuration shown in FIG. 5e, where the second end 332 of the connecting thread 33 is anchored by the anchoring cylinder 32 on the anchoring portion 220 of the outer plate 22 after having traversed the wall P.

Alternatively, it can be arranged so that the abovementioned two axial paths cause compression of the spring 37; then, by releasing the rod 106 from the configuration in FIG. 5d, the feedthrough and anchoring device 3 is returned directly to its retracted position by the release of the spring 37.

At this point, and as shown in FIG. 5f, traction is exerted on the first end 331 of the connecting thread 33 in the direction of arrow F5 by means of the traction device arranged in the applicator 8.

The traction exerted on this first end 331 is transmitted by the thread 33 to the second end 332 anchored on the outer plate 22 and initially causes the support member 23 holding the outer plate 22 to pivot towards the longitudinal axis X, on the distal side of the supporting body 1. As it does so, the outer plate 22 begins to deform the wall P by means of its contact with a portion of the annular surface S surrounding the opening O.

By continuing to exert traction on the first end 331 of the connecting thread 33, this causes the release of the releasable attachment of the outer plate 22 to the support member 23, the upper arms 232 of the support member 23, originally housed in the complementary cavity 222 of the outer plate 22 for example by fitting tightly therein, then exiting said cavity 222.

By pulling even further on the first end 331 of the connecting thread 33, the outer plate 22 deforms the wall P by pressing against the inner plate 11 housed in the supporting body 1; the wall P is then sandwiched between said inner plate 11 and said outer plate 22.

Advantageously, a return prevention device is provided between the connecting thread 33 and the inner plate 11, to prevent the outer plate 22 from relaxing its pressure against the inner plate 11 when the first end 331 of the connecting thread 33 is no longer being pulled. For this purpose, the connecting thread 33 may for example have a beaded portion cooperating with the through-hole 110 provided on the inner plate 11 for the passage of the connecting thread 33, in order to prevent slackening of the portion of thread 33 connecting the outer plate 22 to the inner plate 11.

Subsequently or simultaneously, the deployment mechanism 2, having released the outer plate 22, is returned to its retracted configuration shown in FIG. 5a, the sheath moving in the distal direction.

The applicator 8 is then disconnected from the supporting body 1 and can be removed from the patient's body through the trocar TR.

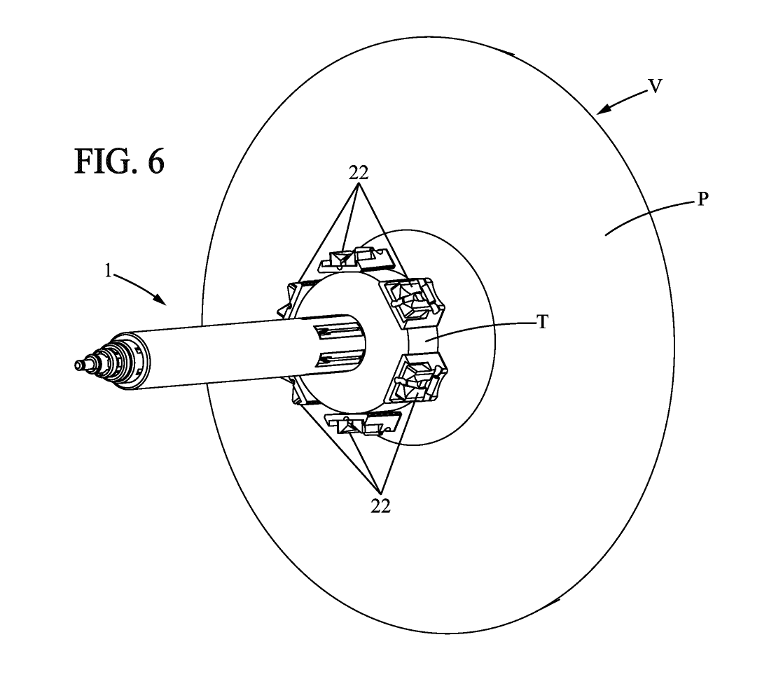

In FIG. 6, all six basic modules of the supporting body 1, to which must be added the six corresponding outer plates, are represented after the step illustrated in FIG. 5f and the disconnection and removal of the applicator 8. FIG. 6 shows a perspective view of the tubular portion T shaped on the wall P around the opening O by means of the preparation system SP in the first embodiment of the invention. This tubular portion T extends here between the outer plates 22 and the cylindrical portion of the supporting body 1 receiving the inner plates 11.

Thus, in this embodiment, the biasing elements which tighten and juxtapose the outer plates 22 on the supporting body, and more specifically on the inner plates 11 supported by the supporting body 1, in order to gradually deform the annular surface S of the wall P centered around the opening O so as to, on the one hand, shape on the organic wall P a tubular portion P extending along the longitudinal axis X, and on the other hand, form an anchoring clamp composed of said outer plates 22, comprise the connecting threads 33 associated with a device for exerting traction on these threads 33.

At this stage one could actuate the ejection device 5 to eject the inner plates 11 from the supporting body, as well as the cutting device 6 to cut the connecting threads 33 in order to be able to remove the supporting body 1 from the patient's body, only leaving within the patient's body the inner and outer plates 11, 22 each connected by a connecting thread 33 of which the second end 332 is secured to an anchoring cylinder 32, all of these elements preferably being made of a bioresorbable material.

The shape of the tubular portion T formed on the wall can then be maintained by the mutual contact of the inner plates 11 which retain each other in a circular configuration, or by a retaining element, for example in the form of a telescoping notched ring, holding the inner plates 11 and/or outer plates 22 in a circular configuration and put in place after removal of the supporting body 1.

One can then see how the anchoring clamp formed by the outer plates 22 could be used to reconnect the formed tubular portion T to an organic or inorganic hollow tube, for example manually by the surgeon.

However, in the context of the present description, the first operative phase of creating the tubular portion is followed by a second operative phase of anastomosis intended to reestablish an end-to-end connection of the patient's urethra to the tubular portion T formed at the created opening on the bladder wall P, by means of the anastomosis system SA proposed by the invention. The ejection device 5 and cutting device 6 are thus only actuated after this reconnection is established, as will be further described below in relation to FIGS. 9, 10a to 10c, 11 and 12.

Alternatively, it could be arranged so that the connecting threads 33 are cut by means of an additional ad hoc tool before advancing to the second operative phase of anastomosis.

Prior to this, the invention proposes a second embodiment of a system SP′ for preparing the bladder wall P to form a tubular portion T which extends from its opening O.

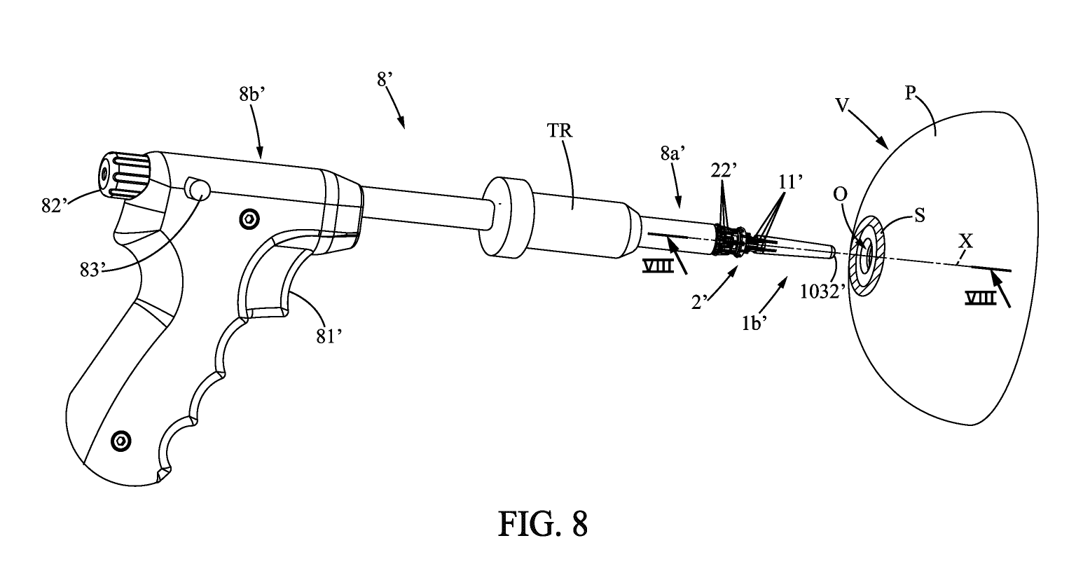

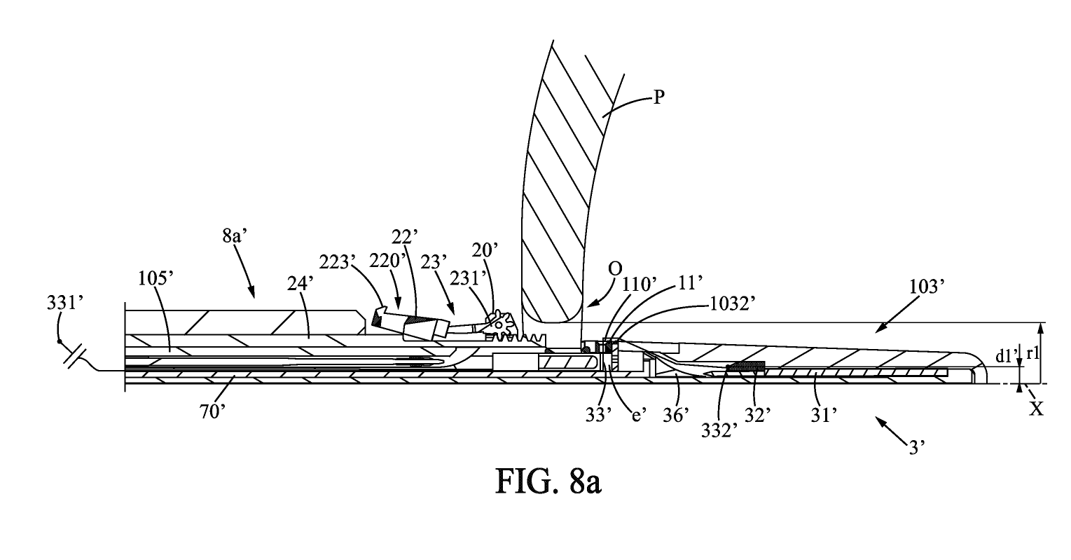

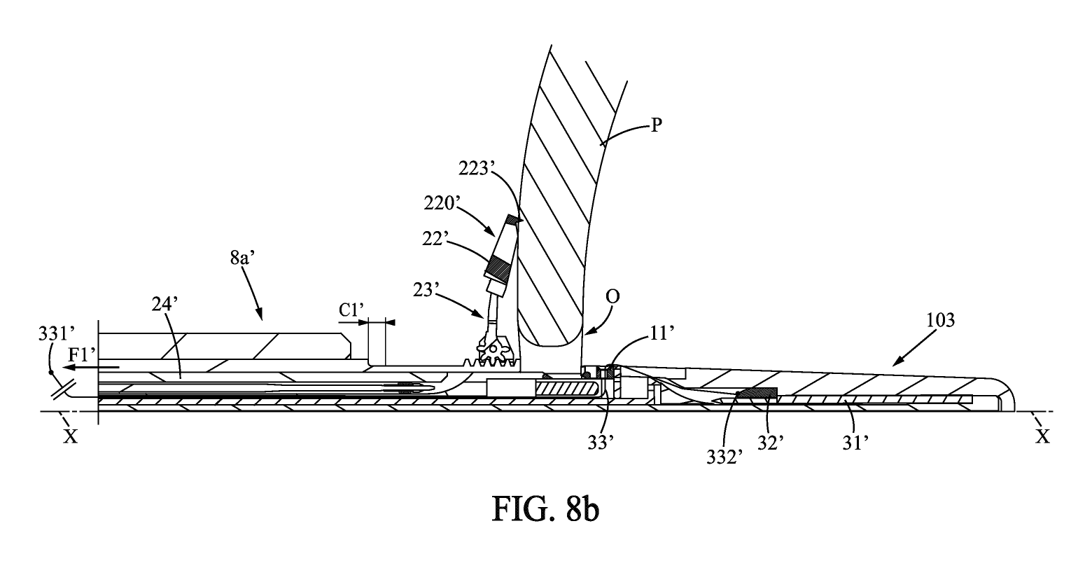

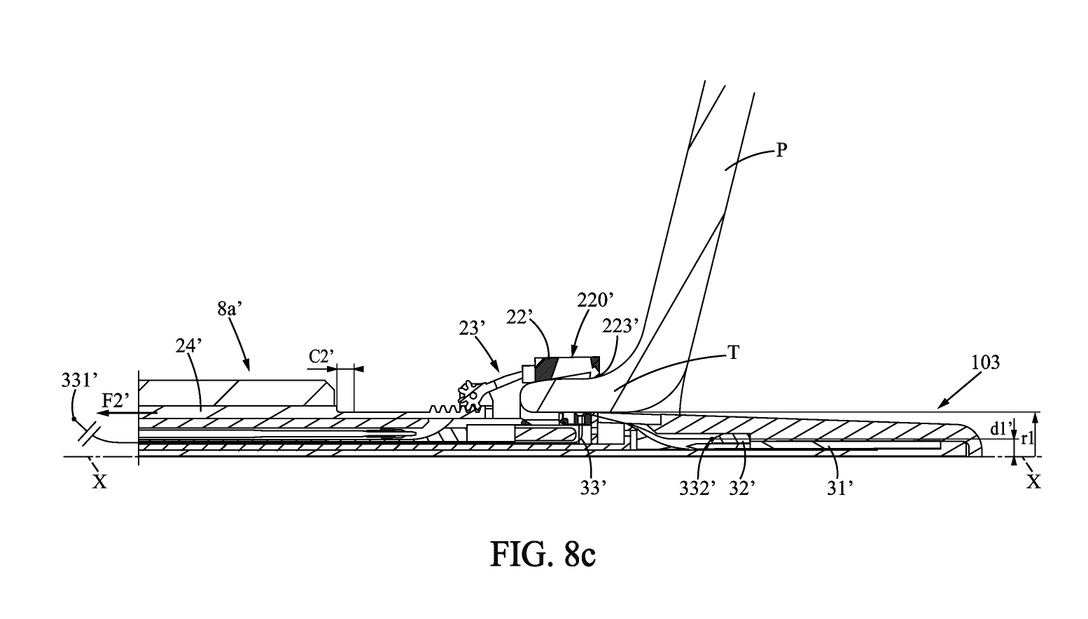

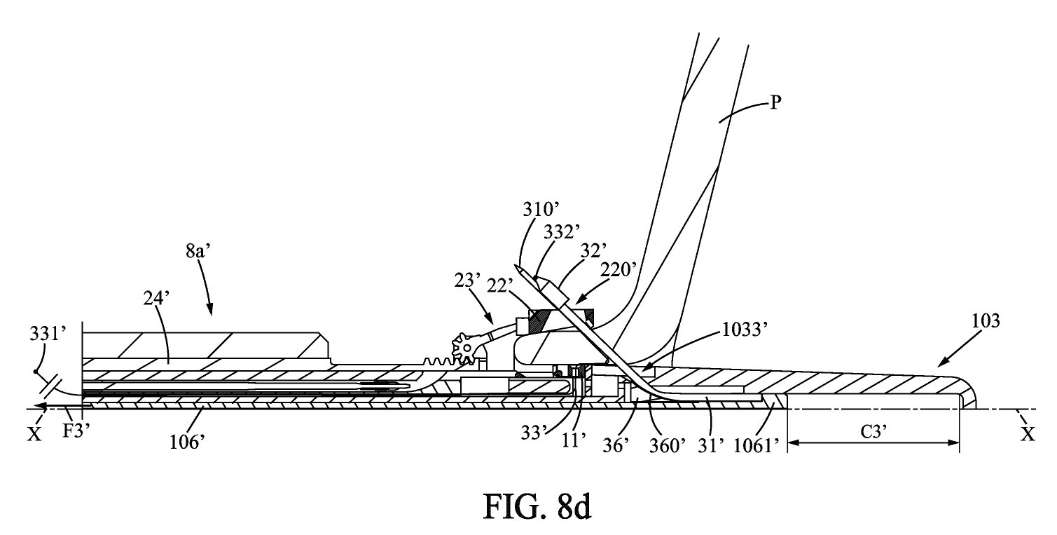

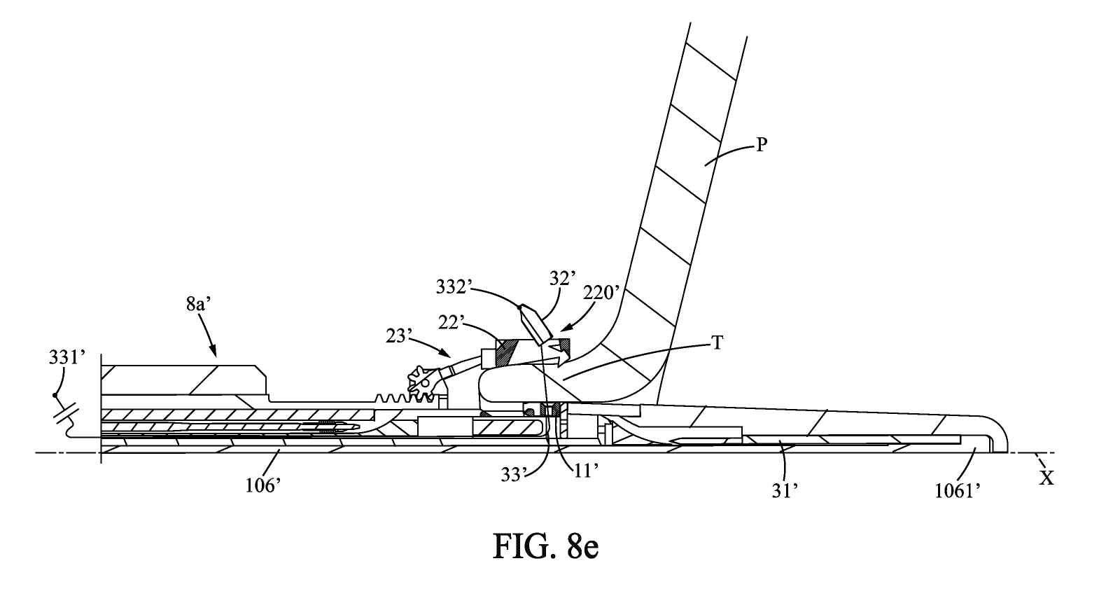

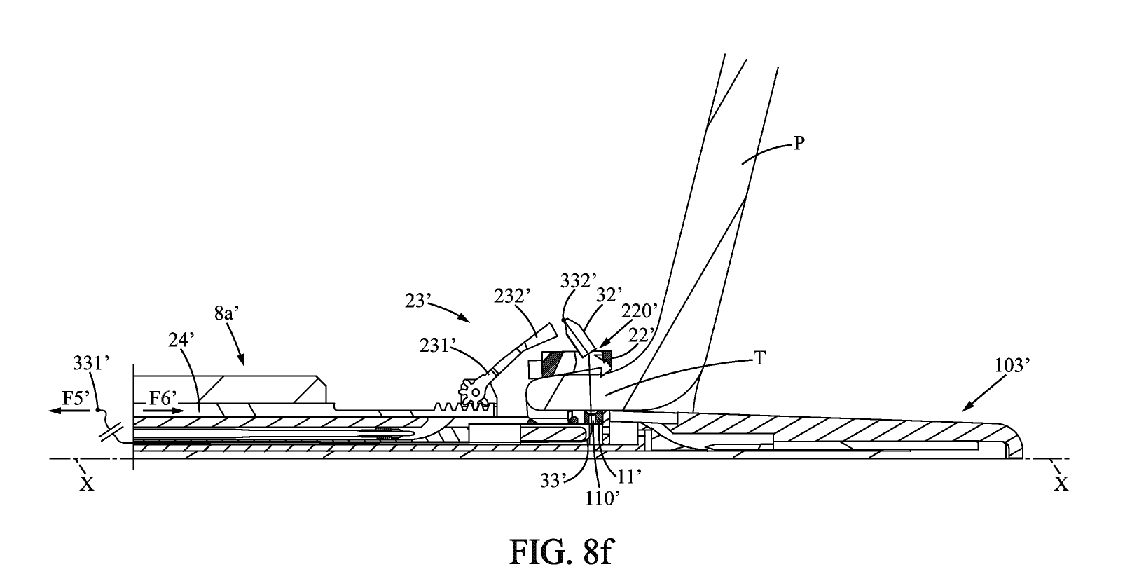

This preparation system SP′ according to the second embodiment makes use of an applicator 8′ identical to the applicator 8 of the first embodiment, except for the deployment device 2′ which differs from the one in the first embodiment. Its operation will be detailed in relation to FIGS. 8a to 8f.

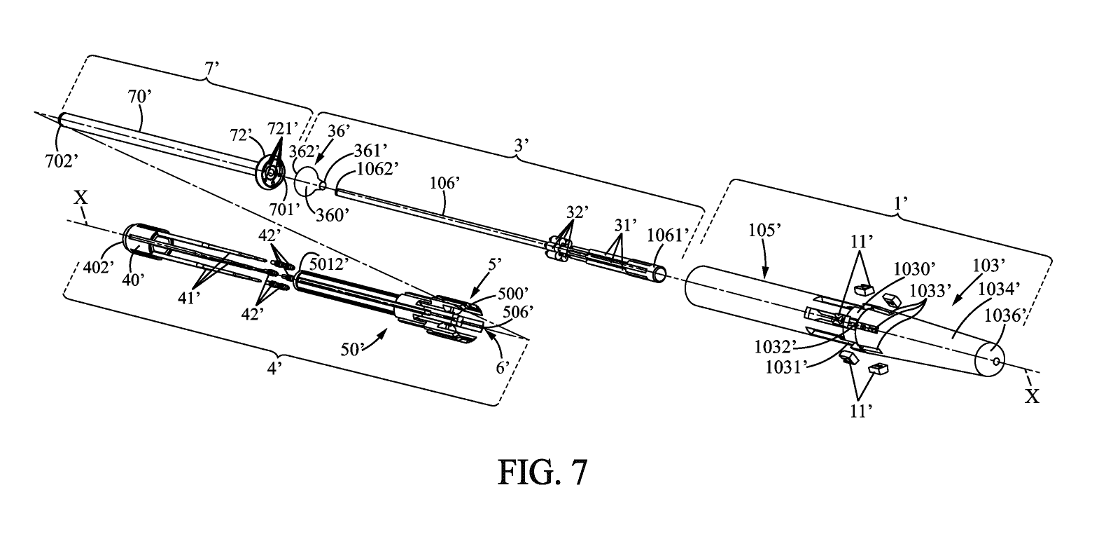

In this second embodiment, and as can be seen in particular in FIG. 7, the supporting body 11 is a single hollow part that is generally cylindrical.

The sleeve 40′, needle 41′, and tiered member 50′ that appear in FIG. 7 are part of a suturing device 4′, and have substantially the same characteristics as the sleeve 40, needle 41, and tiered member 50 described for the first embodiment in relation to FIG. 4; therefore they will not be described further in the context of this second embodiment.

Similarly, the ejection device 5′ and cutting device 6′ visible in FIG. 7 and forming part of the suturing device 4′ are similar to the ejection device 5 and cutting device 6 described for the first embodiment in relation to FIG. 4; therefore they will not be described further in the context of this second embodiment.

For these various elements of the second embodiment, the same references will be used as in the first embodiment, but with the addition of the ′ symbol.

For clarity in FIG. 7, the connecting elements 33′ intended to pass through the wall P of the bladder V, and the suture elements 44′ intended to pass through the urethra, have not been represented; these elements, which are in the form of threads made of bioresorbable materials, are particularly visible in FIGS. 8a to 8f, 10a to 10f, 11 and 12.

Referring to FIG. 7, the supporting body 1′ has a proximal portion 105′ substantially identical to the proximal portion 105 of the fixed part 101 of the supporting body 1 described in detail in relation to FIG. 4, and will not be detailed further in the description of the second embodiment.

In this second embodiment, the supporting body 1′ has a distal portion 103′ comprising a proximal portion 1030′ similar to the proximal portion 1030 of the distal portion 103 described for the first embodiment. This proximal portion 1030′ essentially defines housings 1031′ for receiving inner plates 11′ opening radially outwards and communicating with the inside space of the supporting body so as to provide a space e′ for each associated connecting thread 33′ as is particularly visible in FIG. 8a.

The distal portion 103′ of the supporting body also has a distal portion 1034′ of generally frustoconical shape whose diameter decreases in the distal direction. This frustoconical distal portion 1034′ has a bullet-nose distal end 1036′. The external design of this frustoconical distal portion 1034′ facilitates insertion of the distal portion 103 of the supporting body through the opening O.

This distal portion 103′ also has a plurality of radial through-slots 1033′ that connect the inside space it defines with the outside. The usefulness of these slots 1031′ will be described later.

The supporting body 1′ according to the second embodiment concentrically receives the suturing device 4′, mentioned previously but not described in detail, and a clamping device 7′ and feedthrough and anchoring device 3′.

The feedthrough and anchoring device 3′ is in the form of a rod 106′ extending along the longitudinal axis X and movable in translation along said axis relative to the supporting body 1′. The rod 106′ has a distal shank 1061′ carrying a plurality of resiliently flexible needles 31′ each releasably supporting, on their proximal end, an anchoring element 32′ to which is fixed the second end 332′ of an associated connecting thread 33′. Each anchoring element 32′ is here in the form of a cylinder having a frustoconical proximal end and a housing for receiving the proximal end of the needle 31′ with which it is associated.

The rod 106′ also has a proximal end 1062′ adapted to project proximally beyond the proximal end of the supporting body and intended to be connected to the applicator 8′ for the purposes of controlling the movement of said rod 106′.

The feedthrough and anchoring device 3′ according to this second embodiment further comprises an element 36′ defining a ramp 360′ for the flexible needles 31′; here it is in the form of a rotationally symmetrical element having a distal end 361′ of smaller diameter and a proximal end 362′ of larger diameter. In other words, the diameter of the element 36′ increases gradually in the proximal direction between its distal end 361′ and its proximal end 362′.

This element 36′ is mounted in an integral manner on a distal end 701′ of a mandrel formed in this second embodiment by a hollow shaft 70′ ending at its distal end in an annular flange 72′ having a plurality of through-holes and attachment holes 721′ for the suture threads 44′ (not represented in FIG. 7).

This hollow shaft 70′ is adapted to be threaded onto the rod 106′ and to rotate on said rod about the longitudinal axis. Once in place with the rod 106′ inside the supporting body 1′, the proximal end 702′ of the hollow shaft 70′ protrudes proximally beyond the proximal end of the supporting body 1′, and is intended for connection to the applicator 8′ for the purposes of controlling the shaft 70′.

Finally, again as is visible in FIG. 7, the tiered member 50′ is adapted to be threaded onto the hollow shaft 70′, and the sleeve 40′ supporting the flexible needles 41′ is adapted to be threaded onto the tiered member 50′.

The assembly thus formed comprises the feedthrough and anchoring device 3′, the clamping device 7′, and the suturing device 4′. This assembly is intended for insertion into the supporting body 1′.

Once inserted into the supporting body 1′, the proximal ends 402′, 5012′ of the sleeve 40′ and the tiered member 50′ protrude proximally beyond the proximal end of the supporting body 1′, and are intended to be connected to the applicator 8′ for the purposes of controlling the suturing device 4′.

Thus, in the embodiment illustrated in FIG. 7, the set of elements supported by the supporting body 1′, when they are in their original configurations, fit within a cylinder having a diameter corresponding to the largest outside diameter of the distal portion 103′ of the supporting body 1′. Preferably, this diameter is smaller than the diameter of the opening O present on the wall P, to allow insertion of the distal portion 1a′ of the supporting body through said opening O.

We will now describe a first operative phase of preparing the opening O, arranged on the bladder wall P, so as to form a substantially tubular portion at the opening, making use of the preparation system SP′ according the second embodiment of the invention.

For this purpose, as can be seen in FIG. 8, the proximal portion 1a′ of the supporting body 1′ is inserted along its longitudinal axis X into the housing 8d′ defined in the proximal portion 8a′ of the applicator 8′, and the distal portion 1b′ of the supporting body 1′ extends distally from said proximal portion 8a′ of the applicator 8′.

In this FIG. 8 one can see that, in the second embodiment of the preparation system SP′ of the invention, the distal portion 8a′ of the applicator supports a deployment mechanism 2′ comprising a plurality of support members 23′, visible in particular in FIGS. 8a to 8f, releasably supporting external clamp elements 22′ intended to be positioned facing and then in contact with respective portions of an annular surface S of the wall P centered about the opening O.

This deployment mechanism 2′ is adapted to move the external clamping elements 22′ between a retracted configuration, shown in FIG. 8, where they are folded in the direction of the longitudinal axis X on the proximal end 1a′ side of the supporting body 1′, a deployed configuration where they are away from the longitudinal axis X and flare outward substantially perpendicularly to said axis, and a collapsed configuration where they are folded in the direction of the longitudinal axis X on the distal end side of the supporting body 1′ while deforming the wall P, as will be explained in more detail in relation to FIGS. 8a to 8f.

The distal portion 8a′ of the applicator 8′, equipped with the supporting body 1′ in its initial configuration, and having external clamping elements 22′ in the collapsed configuration, may advantageously be inserted through a trocar TR placed on the abdominal wall of the patient, and the various devices may be controlled from outside the patient's body by means of the proximal portion 8b′ of said applicator 8′, typically during endoscopic surgery.

The applicator 8′ fitted with the supporting body 1′ is then manipulated by the surgeon so that the body 1′ approaches the bladder V, until the distal portion 1a′ of said body is inserted into the opening O formed in the wall P, centering the longitudinal axis X within this opening O and engaging the distal end 1036′ first. The bullet-nose shape of the end 1036′ and the frustoconical shape 1034′ provided on the supporting body 1′ facilitate this insertion.

The external clamping elements 22′, here six in number, are in the form of outer plates made of a bioresorbable material and are releasably supported on the support members 23′, for example by having complementary forms.

The distal portion 8a′ of the applicator 8′ and the proximal portion 1a′ of the supporting body 1′ advantageously have indexing elements which allow fixing said supporting body 1′ to the end of the applicator 8 so that the outer plates 22′, supported by the applicator 8′, are angularly aligned circumferentially with the inner plates 11′ supported by the distal portion 1b′ of the supporting body 1′.

As can be seen in FIGS. 7 and 8, the assembly formed by the distal portion 8a′ of the applicator 8′ and the supporting body 1′ has a rotational symmetry about the longitudinal axis X and can be divided into a plurality of identical basic modules regularly distributed circumferentially. In the example represented, this assembly can be divided into six basic modules each extending over an angular range of about 60° and each comprising an inner plate 11′, an outer plate 22′, a connecting thread 33′ provided for connecting the inner plate 11′ and outer plate 22′ after having passed through the wall P, and a suture thread 44′ provided for connecting the inner plate 11′ and outer plate 22′ after having passed through the urethra.

FIGS. 8a to 8f illustrate one of these modules. The reader will understand that what is visible in these figures also applies to each of the other five modules.

FIGS. 8a to 8f therefore illustrate the various steps in preparing the opening O by means of the preparation system SP′ according to the second embodiment of the invention.

In FIG. 8a, the system SP′ is partially illustrated in its initial configuration when in place at the opening O.

In this initial configuration of the preparation system SP′, the deployment mechanism 2′ is in the configuration with the outer plate 22′ collapsed and supported by a support member 23′, the feedthrough and anchoring device 3′ is in the configuration where the second end 332′ of the connecting thread 33′ is retracted, this end 332′ being at a radial distance d1′ from the longitudinal axis X that is less than the radius r1 of the opening O, the inner plate 11′ is received within its housing 1031′ provided on the supporting body 1′, and the ejection 5′ and cutting device 6′, formed here by a wedge 500′, is in the inactive configuration.

The suturing device 4′ is also in the configuration where the second end 442′ of the suture thread is retracted. This device will come into play during the second operative phase, or anastomosis phase, and its operation will be further described below in relation to FIGS. 10a to 10c and 11. For clarity, the suture thread 44′ is not represented in FIGS. 8a to 8f.

Returning to FIG. 8a, one can see that in this first placement step, the supporting body 1′ is inserted through the opening O until a positioning element 20′ reaches the outer surface of the wall P, or the surface on the proximal side.

In a manner not represented in detail in the figures, the positioning element 20′ is integral with the distal portion 8a′ of the applicator 8′.

In FIG. 8a, one can see that the positioning element 20′ rotatably supports a support member 23′ which is in the form of a generally Y-shaped fork having a lower arm 231′ rotatably mounted on the positioning element 20′ and two upper arms 232′ cooperating with a complementary cavity 222′ provided on the outer plate 22′ to ensure the releasable support of said plate 22′ on the support member 23′. One of these upper arms 232′ and part of the cavity 222′ are visible in FIG. 8f.

The lower arm 231′ has a plurality of peripheral gear teeth, adapted to cooperate with a rack provided on a distal portion of a sheath 24′ mounted to be movable in translation inside the distal portion 8a′ of the applicator 8′ and which can be controlled from the proximal portion 8b′ thereof. In the case of sequential deployment of the external clamping elements, the sheath 24′ can be divided into as many subparts as there are to be successive deployment sequences; each of these subparts is intended to control the deployment of one or more clamping elements between the retracted configuration, the deployed configuration, and the collapsed configuration, and can be successively controlled from the proximal portion 8b′ of the applicator 8′.

In the collapsed configuration of the deployment mechanism 2′, the support 23′ and the outer plate 22′ lie substantially parallel to the longitudinal axis.

Also visible in FIG. 8a is the path of a connecting thread 33′ within the supporting body 1′. This thread 33′ has a first end 331′ adapted for connection to a traction device (not represented) built into the applicator 8′. The thread 33′ traverses the proximal portion 1a′ of the supporting body 1′ within a space provided for this purpose and extends substantially for the length of the hollow shaft 70′. This thread 33′ then emerges into a free space e′ provided in the supporting body 1′ and located under the inner plate 11′ fitted into its housing 1031′. The connecting thread 33′ then passes through the inner plate 11′ by a through-hole 110′, and changes direction again as it follows the outer surface of the plate 11′; then it passes through the notch 1032′ and is fixed by its second end 332′ to an anchoring element 32′ releasably supported by a flexible needle 31′.

Referring now to FIG. 8b, the controls have moved the sheath 24′ along an axial path C1′ in the direction of arrow F1′, which has caused the support member 23′ to pivot due to the engagement of its teeth with the rack supported by the sheath, resulting in a deployed configuration of the outer plate 22′ where it lies substantially perpendicular to the longitudinal axis X, facing a portion of the annular surface S of the wall P visible in FIG. 7.

Advantageously, in this deployed position, at least one catch hook or tooth 223′ provided on the outer plate 22′ catches on a portion of the annular surface S of the wall P visible in FIG. 7.

Turning to FIG. 8c, the sheath 24′ has again been moved by appropriate controls along an axial path C2′ in the direction of arrow F2′, which has caused the support member 23′ to pivot due to the engagement of its teeth with the rack supported by the sheath, resulting in a collapsed configuration of the outer plate 22′ where it is folded in the direction of the longitudinal axis X on the distal end side of the supporting body. The tooth 223′ having caught on the wall P, as the outer plate 22′ folds it pulls along a portion of the annular surface S of the wall P and deforms it so that it is pressed against the supporting body, and more specifically against the inner plate 11′.

Thus, in this second embodiment, the biasing elements arranged to tighten and juxtapose the outer plates 22′ on the supporting body 1′, and more precisely on the inner plates 11′ supported by the supporting body 1′, in order to progressively deform the annular surface S of the wall P centered around the opening O so as to, on the one hand, shape on the organic wall P a tubular portion P extending along the longitudinal axis X, and on the other hand, form an anchoring clamp consisting of said outer plates 22′, comprise the deployment mechanism 2′.

FIG. 8d illustrates a subsequent step in the operation of the preparation system SP′ according to the second embodiment of the invention. During this step, the intent is to maintain or lock in the configuration shown in FIG. 8c the outer plate 22′ and inner plate 11′ gripping the wall P, by means of a connecting thread 33′ linking said inner and outer plates 11′, 22′.

For this purpose, the feedthrough and anchoring mechanism 3′ is actuated so that the second end 332′ is moved from its retracted configuration where it is positioned at a radial distance d1′ from the longitudinal axis X, visible for example in FIG. 8c, to its anchored configuration where it is retained on the outer plate 22′ after having traversed the wall P.

This actuation is achieved by moving the rod 106′ in the direction of arrow F3′ along a path C3′. This displacement of the rod 106′ drives the flexible needle 31′ in axial translation by means of the shank 1061′. As it comes in contact with the ramp 360′ provided on the element 36′, the flexible needle 31′ bends radially outward, passes through a through-slot 1033′ arranged in the supporting body 1′, and obliquely traverses the tubular portion T formed on the wall P, as well as the anchoring portion 220′ provided on the outer plate 22′.

The path C3′ of movement of the rod 106′ will be dimensioned appropriately to allow the second end 332′ of the connecting thread 33′ and the anchoring cylinder 32′ which supports it to pass entirely through the wall P and outer plate 22′ by passing through the anchoring portion 220′.

For this purpose, in the embodiment illustrated, the needle 31′ has a tip 310′ for piercing the wall P, on its proximal end.

When the rod 106′ is released, the configuration illustrated in FIG. 8e is reached, where the feedthrough and anchoring device 2′ returns to its retracted configuration; as the distal end of the needle 31′ is no longer subjected to force from the shank 1061′ of the rod, said needle 31′ returns naturally by elastic return to its rest position where it lies straight and substantially parallel to the longitudinal axis X. In doing so, it presses against the ramp 360′ and slides thereon while transmitting this return movement to the rod 106′ through its distal end integral with the shank 1061′.

During this return movement, the needle 31′ releases the anchoring cylinder 32′ to which the second end 332′ of the connecting thread 33′ is attached, this cylinder 32′ then catching on the anchoring portion 220′ of the outer plate 22′.

At this point, and as shown in FIG. 8f, traction is exerted on the first end 331′ of the connecting thread 33′ in the direction of arrow F5′ by means of the traction device arranged in the applicator 8′.

The traction exerted on this first end 331′ is transmitted by the thread 33′ to the second end 332′ anchored on the outer plate 22′ and causes the outer plate 22′ to press more tightly against the inner plate 11′, sandwiching the wall P between them.

Advantageously, a return prevention device is provided between the connecting thread 33′ and the inner plate 11′, to prevent the outer plate 22′ from relaxing its pressure against the inner plate 11′ when the first end 331′ of the connecting thread 33′ is no longer being pulled. For this purpose, the connecting thread 33′ may for example have a beaded portion cooperating with the through-hole 110′ provided on the inner plate 11′ for the passage of the connecting thread 33′, in order to prevent slackening of the portion of thread 33′ connecting the outer plate 22′ to the inner plate 11′.

Subsequently or simultaneously, the sheath 24′ is moved in the distal direction along arrow F6′ so as to cause the support member 23′ to pivot to a retracted position in the direction of the longitudinal axis X, on the proximal side of the supporting body 1′. This pivoting causes the release of the releasable attachment of the outer plate 22′ to the support member 23′, the upper arms 232′ of the fork formed by said support member 23′ exiting the complementary cavity 222′ provided on the outer plate 22.

The applicator 8′ is then disconnected from the supporting body 1′ and can be removed from the patient's body through the trocar TR.

Thus, in this second embodiment, the biasing elements arranged to tighten and juxtapose the outer plates 22′ on the supporting body 1′, and more specifically on the inner plates 11′ supported by the supporting body 1′, in order to gradually deform the annular surface S of the wall P centered around the opening O so as to, on the one hand, shape on the organic wall P a tubular portion T extending along the longitudinal axis X, and on the other hand, form an anchoring clamp composed of said outer plates 22′, comprise the connecting threads 33′ associated with a device for exerting traction on these threads 33′, in addition to the deployment mechanism 2′.

At this stage one could actuate the ejection device 5′ to eject the inner plates 11′ from the supporting body 1′, as well as the cutting device 6′ to cut the connecting threads 33′ in order to be able to remove the supporting body 1′ from the patient's body, only leaving inside the patient's body the inner and outer plates 11′, 22′ each connected by a connecting thread 33′ of which the second end 332′ is secured to an anchoring cylinder 32′, all of these elements preferably being made of a bioresorbable material.

The shape of the tubular portion T formed on the wall can then be maintained by the mutual contact of the inner plates 11′ which retain each other in a circular configuration, or by a retaining element, for example in the form of a telescoping notched ring, holding the inner 11′ and/or outer plates 22′ in a circular configuration and put in place after removal of the supporting body 1′.

One can then see how the anchoring clamp formed by the outer plates 22′ could be used to reconnect the formed tubular portion T to an organic or inorganic hollow tube, for example manually by the surgeon.

However, in the context of the present description, the first operative phase of creating the tubular portion is followed by a second operative phase of anastomosis intended to reestablish an end-to-end connection of the patient's urethra to the tubular portion T formed at the created opening on the bladder wall P, by means of the anastomosis system 1 proposed by the invention.

In this second operative phase, the applicator 8, 8′ has been disconnected from the supporting body 1, 1′ and removed from the patient's body. The surgeon uses the probe 9, which is identical in the two embodiments described above. The elements used during this second operative phase, or anastomosis phase, are substantially identical in either of the embodiments described.

This anastomosis or reconnection phase will therefore be described in relation to FIGS. 9, 10a to 10b, 11 and 12, where the preparation system SP according to the first embodiment is used, although it would apply as same manner using the described preparation system SP′ according to the second embodiment.

With reference to FIG. 9, after disconnecting and removing the applicator, the surgeon makes use of the probe 9. The probe 9 is inserted into the urethra C from its meatus C, until it reaches the opposite end E visible in FIG. 9. It is understood that this end E results from a prior resection of the membranal area of the urethra, in particular when carried out as part of a prostatectomy.

As illustrated in FIG. 9, the probe 9 is inserted into the urethra so that the distal end 9a is positioned at the end E of the urethra C. To facilitate this positioning, the distal end 9a of the probe is preferably provided with a domed cap which, once the probe is positioned, is removed by the surgeon by an additional ad hoc tool.

The surgeon then brings together the tubular portion T formed on the bladder wall P and the end E of the urethra C. In practice, and in particular to prevent damage to the sphincter of the urethra C, preferably it is the wall P of the bladder V which is acted upon to bring its tubular portion T toward the end E of the urethra C. To achieve this, the surgeon can pull on the supporting body 1 or on the outer plates 24 as indicated by arrow F in FIG. 9 in order to close the gap between the two organs to be anastomosed so that the mucous membranes of their respective edges are axially facing one another.

The surgeon then connects the distal end 9a of the probe 9 to the proximal portion of the supporting body 1, the connecting and control elements (not represented) engaging with the proximal end 702 of the hollow shaft 70, the proximal end 5012 of the tiered member 50, and the proximal end 402 of the support sleeve 40 of the flexible needles 41.

The distal portion 9a of the probe 9 and the proximal portion 1a of the supporting body 1 advantageously have indexing elements which allow attaching said supporting body 1 to the end of the probe 9 such that the flexible needles 41, supported by the supporting body 1, are angularly aligned circumferentially with the through-slots 95 provided on the distal portion 9a of the probe 9.

Actuation of the suturing device 4, bringing into play a plurality of suture elements 44, will now be described with reference to FIGS. 9 and 10a to 10c.

In FIG. 9, the suturing device 4 is in the configuration where the second ends 442 of the suture elements 44 are retracted, said suture elements here being in the form of threads, preferably made of a bioresorbable material.

In this retracted configuration, the flexible needles 41, releasably supporting hollow cylinders 42 to which are fixed the second ends 442 of the suture threads 44, are received within longitudinal channels 504 provided in the tiered member 50 and visible in particular in FIG. 4. The second ends 442 of the suture threads 44 are then located at a radial distance d2 (visible in FIG. 10c) which is less than the inside radius r2 of the urethra C.

In FIGS. 10a to 10c, only one basic module has been represented. It is understood that the description given in relation to this basic module applies to the other five, which are evenly distributed circumferentially.

Thus, FIG. 10a illustrates the suturing device 4 after the flexible needle 41 has been moved in the direction of arrow F6 by acting on the proximal end 402 of the support sleeve 40.

During this axial movement, the distal end of the needle 41 comes in contact with the ramp 505 provided on the tiered member 50, at the distal end 504 of the channel in which it was completely housed in the initial configuration, this ramp which is part of the tiered member 50 initially being maintained in a fixed manner in the axial direction, for example by means of the probe, in a first axial position

The flexible needle 41 is then bent radially outward so as to pass through a through-slot 1051 arranged in the proximal portion 105 of the fixed part 101 of the supporting body 1, then through a through-slot 95 arranged in the distal portion 9a of the probe 9, and then passes at an angle through the end E of the urethra C.

The axial travel of the sleeve 40 will be dimensioned appropriately for the second end 442 of the suture thread 44 and the anchoring cylinder 42 that supports it to pass entirely through the end E of the urethra C.

For this purpose, in the embodiment illustrated, the needle 41 has a tip 410 for piercing the wall P, on its proximal end.

In the position illustrated in FIG. 10a, the suturing device 4 is in an intermediate deployed configuration, where the second end 442 of the suture thread 44 is positioned facing an anchoring portion 224, visible for example in FIG. 9, provided on the associated outer plate 22.

In this FIG. 10a, one can also see the path of the suture thread 44. This thread 44 has a first end 441 connected distally behind an opening 721 provided on the mandrel 72. The thread 44 then runs in the distal direction and successively passes through the opening 721, an opening 711 formed on the annular ring 71 integral with the supporting body 1, then passes through the inner plate 11 via a passage 120 provided for this purpose in said plate. The thread 44 then reaches the flexible needle 41 and runs along it until it reaches the anchoring cylinder 42, to which its second end 442 is attached.

In the retracted configuration of the suturing device, the needle 41 is housed within its associated channel 504, and the thread 44 exits the passage 120 on the distal side of the inner plate 11, runs within the through-slot 507 formed in the wedge 500, and emerges into the channel 504 at the ramp 505.

Referring now to FIG. 10b, the tiered member 50, of which the ramp 505 is a part, has been moved in the direction of arrow F7 by acting on its proximal end 5012 connected to the probe 9, in order to reach a second axial position. As the flexible needle 41 is no longer in contact with the ramp 505, it folds back by elastic return towards the longitudinal axis; by doing so, the needle 41 slightly deforms the end E of the urethra C and anchors the anchoring cylinder 42 supporting the second end 442 of the suture thread inside the complementary anchoring portion 224 provided on the outer plate 22.

At this point, the needle 41 is moved in the direction of arrow F8 illustrated in FIG. 10c so that it returns to its original configuration inside its associated channel 504, having released the anchoring cylinder 42 carrying the second end of the suture thread 44 inside the anchoring portion 224 provided in the outer plate 22.

Subsequently or simultaneously, the mandrel 72 is rotatably driven via the hollow shaft 70 connected at its proximal end 702 to the probe 9. This mandrel 72 then pulls on the first end 441 of the suture thread 44 and winds it around the body of the mandrel 72. By doing so, the end-to-end anastomosis between the tubular portion T of the wall P of the bladder V and the end E of the urethra C is pulled tighter.

Advantageously, the mandrel 72 is associated with a torque limiting system, arranged in the probe 9 controlling the rotation of the hollow shaft 70 integral with the mandrel 72, or directly by a torque limiting device, for example a device with calibrated slip/friction arranged between said shaft 70 and said mandrel 72. The tightness of the anastomosis can thus be adjusted in a precise and reproducible manner.

Advantageously, a return prevention device is provided between the suture thread 44 and the inner plate 11, this device preventing slackening of the thread 44 and of the anastomosis when the first end 441 of the suture thread 44 is no longer being pulled. For this purpose, the suture thread 44 may, for example, have a beaded portion cooperating with the passage 120 provided on the inner plate 11 for the passage of the suture thread 44, in order to prevent slackening of the portion of thread 44 connecting the outer plate 22 to the inner plate 11 via the urethra C.

Once the anastomosis between the patient's urethra C and bladder V has been established and tightened, FIG. 11 illustrates a step of ejecting the inner plate 11 and cutting the connecting 33 and suture 44 threads.

To do this, the tiered member 50 is moved axially in the direction of arrow F9 in FIG. 11, until the ejection face 509 of the wedge 500 comes in contact with the inner plate 11. As the ejection face 509 is sloped, this moves the inner plate 11 in the radial direction so that it emerges from its housing 1031 provided in the supporting body 1. During this radial displacement, the inner plate 11 slightly compresses the bladder wall P at the tubular portion T as is clearly visible in FIG. 11.

Continuing its forward movement in the direction of arrow F9, the cutting blade 506 of the wedge 500 engages with the suture thread 44 and connecting thread 33 then catches them and cuts them against a radial face, which is for example part of the annular ring 71 integral with the supporting body 1.

As the inner plate 11 has been ejected from the supporting body and the connecting 33 and suture 44 threads have been cut behind the inner plate 11, the supporting body 1 can be removed from the work area and from the patient's body, through the probe.

Thus, if we consider the set of six basic modules, the elements remaining within the patient's body are limited to six inner plates 11, six outer plates 22, six connecting threads 33 each having an anchoring cylinder 32 at their end, and six suture threads 44 each having an anchoring cylinder 42 at their end, all these elements being preferably made of a bioresorbable material.

Finally, the operation of the system for performing anastomosis between the bladder wall P and the urethra C, according to the second object of the invention and for the case where a tubular portion is formed on the organic wall by a means other than the preparation system mentioned above, avoids a number of steps described for the first object of the invention.

In particular, the steps related to the deployment and placement of external clamping elements 22, 22′ on the wall P in order to form the tubular portion T do not apply to this second object.

Conversely, the description provided in relation to FIGS. 8d to 8f, 9, 10a to 10c, 11 and 12, detailing the various phases of feeding through and anchoring the connecting and suture threads 33′, 44′, as well as the phases of tightening these threads and the phases of ejecting the inner plates 11′ and cutting the threads 33′, 44′, apply to the system according to this second object.

Different options can be considered for defining a tubular portion on the wall P, making use of one or more external clamping elements 22. One could, for example, provide only one external clamping element in the form of an elastically deformable ring having a plurality of anchoring portions uniformly distributed along its circumference that are intended to accommodate the anchoring elements for the connecting and suture threads, and put in place by slipping it around the flattened wall P, for example manually over a cylindrical portion of the supporting body. When placed in this manner, the ring would serve as an anchoring clamp for the connecting and suture threads.

1. A system for preparing an opening created on an organic wall, comprising:
a supporting body defining a longitudinal axis and having a proximal portion and a distal portion, said distal portion being adapted for insertion through the opening;
external clamping elements deployable in an outwardly flaring arrangement relative to the supporting body while being distributed circumferentially, and able to cover, about said opening, portions of an annular surface of said organic wall, and;
biasing elements arranged to tighten and juxtapose said external clamping elements on said supporting body in order to progressively deform said annular surface so as to, on the one hand, shape on said organic wall a tubular portion extending along the longitudinal axis, and on the other hand, form an anchoring clamp consisting of said external clamping elements.

2. The system according to claim 1, wherein the biasing elements comprise connecting elements, each connecting element being adapted to connect an external clamping element to the supporting body, and a device for exerting traction on the connecting elements, said connecting elements each having a first end connected to said traction device and a second end adapted to be retained on an associated external clamping element, and actuation of said traction device tightening and juxtaposing said external clamping elements on said supporting body.

3. The system according to claim 2, further comprising a feedthrough and anchoring device adapted to move the second end of each connecting element between a retracted configuration where said second end is located at a radial distance from the longitudinal axis that is less than the radius of the opening, and an anchored configuration where said second end is retained on an associated external clamping element after passing through the wall.

4. The system according to claim 1, further comprising a deployment mechanism adapted to move said external clamping elements at least between a retracted configuration where they are folded in the direction of the longitudinal axis, and a deployed configuration where they are away from the longitudinal axis and flare outwards substantially perpendicularly to it;
and wherein the deployment mechanism comprises support members on which the external clamping elements are releasably supported.

5. The system according to claim 4, wherein the deployment mechanism is further adapted to move the external clamping elements between the deployed configuration and a collapsed configuration where said external clamping elements are folded in the direction of the longitudinal axis on the distal end side of the supporting body;
and wherein said deployment mechanism is part of the biasing elements.

6. The system according to claim 5, wherein the feedthrough and anchoring device is adapted to move the second end of each connecting element between the retracted configuration and the anchored configuration when the external clamping elements are in the collapsed position.

7. The system according to claim 3, wherein the feedthrough and anchoring device is adapted to move the second end of each connecting element between the retracted configuration and the anchored configuration when the external clamping elements are in the deployed configuration.

8. The system according to claim 1, wherein the supporting body releasably supports internal clamping elements angularly aligned circumferentially with the external clamping elements,
and wherein the biasing elements are arranged to tighten and juxtapose said external clamping elements on said internal clamping elements.

9. The system according to claim 8, further comprising an ejection device adapted to move the internal clamping elements between a retracted configuration where they are at least partially housed within the supporting body, and an ejected configuration where they are removed from said supporting body.

10. The system according to claim 2, wherein each connecting element comprises a portion associated with a respective internal clamping element, the system further comprising a cutting device adapted for cutting the connecting elements between the internal clamping elements and the first ends of said connecting elements.

11. The system for performing an anastomosis between an organic wall having an opening and an organic hollow tube, said system comprising:
the system according to claim 8, and
a device for connecting said tube and the tubular portion shaped on the wall, said device being arranged at least partially within a proximal portion of the supporting body, said proximal portion being adapted for insertion into the tube and comprising:
suture elements adapted for connecting the internal and external clamping elements and each having a first end connected to a tightening device and a second end adapted to be retained on an associated external clamping element; and
a suturing device adapted for moving the second end of each suture element between a retracted configuration where said second end is positioned at a radial distance from the longitudinal axis that is less than the inside radius of said tube, and an anchored configuration where said second end is retained on an associated external clamping element after passing through the tube from the inside to the outside.

12. The system according to claim 11, wherein each suture element comprises a portion associated with a respective internal clamping element, the system further comprising a cutting device adapted for cutting the suture elements between the internal clamping elements and the first ends of said suture elements.

13. A method for preparing an opening created on an organic wall, wherein:
a supporting body defining a longitudinal axis and having a proximal portion and a distal portion is provided;
external clamping elements are also provided;
the distal portion of the supporting body is introduced through the opening;
the external clamping elements are deployed so as to flare outward relative to the supporting body, said external clamping elements being distributed circumferentially and covering, around said opening, portions of an annular surface of said organic wall;
the external clamping elements are urged so as to tighten and juxtapose said external clamping elements on said supporting body in order to progressively deform said annular surface so as to, on the one hand, shape on said organic wall a tubular portion extending along the longitudinal axis, and on the other hand, form an anchoring clamp consisting of external clamping elements.

14. The method for preparing an opening arranged on an organic wall according to claim 13, wherein, the supporting body releasably supporting internal clamping elements angularly aligned circumferentially with the external clamping elements, the external clamping elements are urged so as to tighten and juxtapose said external clamping elements on said internal clamping elements.

15. The method for performing an anastomosis between an organic wall having an opening and an organic hollow tube, wherein:
the opening created on the wall is prepared in accordance with the method of claim 14;
the proximal portion of the supporting body is introduced at least partially into the tube;
suture elements are provided, adapted for connecting the internal and external clamping elements and each having a first end connected to a tightening device and a second end adapted to be retained on an associated external clamping element; and
the second end of each suture element is moved between a retracted configuration where said second end is located at a radial distance from the longitudinal axis that is less than the inside radius of said tube, and an anchored configuration where said second end is retained on an associated external clamping element after passing through the associated tube from the inside to the outside.

16. The method for performing an anastomosis according to claim 15, wherein, each suture element comprising a portion associated with a respective internal clamping element, the suture elements are cut between the internal clamping elements and the first ends of said suture elements.

17. The system according to claim 8, wherein each connecting element comprises a portion associated with a respective internal clamping element, the system further comprising a cutting device adapted for cutting the connecting elements between the internal clamping elements and the first ends of said connecting elements.
